(19) United States
(12) Patent Application Publication
(10) Pub. No.: US 2024/0175051 A1
(43) Pub. Date: May 30, 2024

(54) CD47 COMPOSITIONS AND METHODS FOR THE TREATMENT OF DEGENERATIVE OCULAR DISEASES

(71) Applicant: President and Fellows of Harvard College, Cambridge, MA (US)

(72) Inventors: Sean K. Wang, Cambridge, MA (US); Constance L. Cepko, Cambridge, MA (US)

(21) Appl. No.: 18/382,626

(22) Filed: Oct. 23, 2023

Related U.S. Application Data
(63) Continuation of application No. PCT/US2022/026100, filed on Apr. 25, 2022.
(60) Provisional application No. 63/179,753, filed on Apr. 26, 2021.

(51) Int. Cl. C12N 15/86; A61K 48/00; A61P 27/02

(52) U.S. Cl. CPC C12N 15/86; A61K 48/005; A61P 27/02; C12N 2750/14143

(57) ABSTRACT

The present invention provides compositions, e.g., pharmaceutical compositions, which include a recombinant adeno-associated viral (AAV) expression construct, AAV vectors, AAV particles, and methods of treating a subject having a degenerative ocular disorder, e.g., retinitis pigmentosa.

### RELATED APPLICATIONS

This application is a 35 § U.S.C. 111(a) continuation application which claims the benefit of priority to PCT/US2022/026100, filed on Apr. 25, 2022, which, in turn, claims the benefit of priority to U.S. Provisional Application No. 63/179,753, filed on Apr. 26, 2021. The entire contents of the foregoing applications are incorporated herein by reference.

### SEQUENCE LISTING

The instant application contains a Sequence Listing which has been submitted electronically in XML file format and is hereby incorporated by reference in its entirety. Said XML copy, created on Nov. 10, 2023, is named 117823-31902_SL.xml and is 124,696 bytes in size.

### BACKGROUND OF THE INVENTION

Inherited retinal diseases (IRDs) are a group of diseases that can cause severe vision loss or even blindness. Each IRD is caused by at least one gene that is not working as it should. Retinitis pigmentosa (RP), the largest family of these disorders, is a disease of the eye that presents with progressive degeneration of rod and cone photoreceptors, the light-sensing cells of the retina (Hartong D T, et al. (2006) Lancet 368(9549):1795-1809). The disease can result from mutations in any of over 100 different genes and is the most common inherited form of blindness in the world, affecting an estimated 1 in 4000 individuals (Daiger S P, et al. (2013) Clin Genet 84(2):132-141; Berson E L (1996) Proc Natl Acad Sci USA 93(10):4526-8; Haim M (2002) Acta Ophthalmol Scand Suppl (233):1-34).

A major obstacle in developing treatments for IRDs is the enormous genetic heterogeneity of pathogenic mutations. One approach to treat this group of diseases is gene therapy, e.g., using adeno-associated vectors (AAVs) to deliver a wild-type allele to complement a mutated gene (Ali R R, et al. (1996) Hum Mol Genet 5(5):591-4; Murata T, et al. (1997) Ophthalmic Res 29(5):242-251). While this approach has proven successful in other conditions, even leading to the approval of a gene therapy for RPE65-associated Leber's congenital amaurosis (Maguire A M, et al. (2008) N Engl J Med 358(21):2240-2248), it is difficult to implement for the majority of RP patients, given the extensive heterogeneity of genetic lesions (Daiger S P, et al. (2013) Clin Genet 84(2):132-141). A broadly applicable gene therapy that is agnostic to the genetic lesion would provide a treatment option for a greater number of RP patients. Presently, there is no effective therapy of any kind for RP, and despite more than a dozen randomized clinical trials to date, none have been able to demonstrate an improvement in visual function (Sacchetti M, et al. 2015) J Ophthalmol 2015:737053).

In patients with RP, there is an initial loss of rods, the photoreceptors that mediate vision in dim light. Clinically, this results in the first manifestation of RP, poor or no night vision, which usually occurs between birth and adolescence (Hartong D T, et al. (2006) Lancet 368(9549):1795-1809). Daylight vision in RP is largely normal for decades, but eventually deteriorates beginning when most of the rods have died. This is due to dysfunction, and then death, of the cone photoreceptors, which are essential for high acuity and color vision. Loss of cone function is the major source of morbidity in the disease (Hartong D T, et al. (2006) Lancet 368(9549):1795-1809). Importantly, while the vast majority of genes implicated in RP are expressed in rods, few actually exhibit expression in cones, suggesting the existence of one or more common mechanisms by which diverse mutations in rods trigger non-autonomous cone degeneration (Narayan D S, et al. (2016) Acta Ophthalmol 94(8):748-754; Wang W, et al. (2016) Cell Rep 15(2):372-85; Komeima K, et al. (2006) Proc Natl Acad Sci USA 103(30):11300-5).

Accordingly, there is a need in the art for therapies to treat and prevent vision loss that results from degenerative retinal diseases, such as RP.

### SUMMARY OF THE INVENTION

The present invention is based, at least in part on the discovery of mutation-independent compositions and methods of treatment for subjects having a degenerative disease, e.g., a degenerative ocular disease, such as retinitis pigmentosa (RP).

It has surprisingly been discovered that augmentation of CD47-SIRPα signaling could promote cone survival in RP-mutant mice. More specifically, the inventors have successfully developed an adeno-associated viral (AAV) vector expressing CD47 (AAV8-RedO-CD47), and have surprisingly discovered that delivery of this vector expressing CD47 in cones promoted cone survival and helped preserve visual function in multiple RP-mutant mice, and that this protection mediated by CD47 requires SIRPα.

It has also surprisingly been discovered that delayed expression of CD47 could also preserve cones, demonstrating that this therapy would be beneficial even if administered later in disease, after most rods have died. Therefore, augmentation of CD47-SIRPα signaling by, for example, delivery of AAV8-RedO-CD47, offers a potential treatment option for many patients with RP, including those who are diagnosed at older age or with genetics that preclude straightforward gene replacement. This approach may further help combat cone death in other inherited retinal diseases and degenerative retinal disorders. Moreover, augmentation of CD47-SIRPα signaling could also serve as a treatment strategy for other forms of neurodegeneration disorders, such as age-related macular degeneration (AMD), multiple sclerosis, glaucoma or amyotrophic lateral sclerosis (ALS).

Accordingly, the present invention provides compositions, e.g., pharmaceutical compositions, which include an agent that enhances CD47-SIRPα signaling, e.g., a recombinant adeno-associated virus (AAV) vector expressing CD47, and methods of treating a subject having a degenerative ocular disorder, e.g., retinitis pigmentosa.

In one aspect, the present invention provides a method for treating or preventing a degenerative ocular disorder in a subject. The method comprises administering to the subject a therapeutically effective amount of an agent that enhances CD47-SIRPα signaling, thereby treating or preventing the degenerative ocular disorder in the subject.

In another aspect, the present invention provides a method for preventing loss of functional vision in a subject having a degenerative ocular disorder. The method comprises administering to the subject a therapeutically effective amount of an agent that enhances CD47-SIRPα signaling, thereby preventing loss of functional vision in the subject.

In another aspect, the present invention provides a method for prolonging the viability of a photoreceptor cell compromised by a degenerative ocular disorder. The method comprises contacting the cell with an agent that enhances CD47-SIRPα signaling, thereby prolonging the viability of the photoreceptor cell compromised by the degenerative ocular disorder.

In some embodiments, the contacting occurs in vitro.

In some embodiments, the cell is within a subject.

In some embodiments, the subject is a human subject.

In some embodiments, the degenerative ocular disorder is associated with decreased viability of cone cells and/or decreased viability of rod cells.

In some embodiments, the degenerative ocular disorder is selected from the group consisting of retinitis pigmentosa, age related macular degeneration, cone rod dystrophy, rod cone dystrophy and glaucoma.

In some embodiments, the degenerative ocular disorder is a genetic disorder. In some embodiments, the degenerative ocular disorder is not associated with blood vessel leakage and/or growth. In some embodiments, the degenerative ocular disorder is retinitis pigmentosa.

In some embodiments, the agent prevents degeneration of a cone photoreceptor cell.

In some embodiments, the agent increases the expression and/or activity of CD47 and/or SIRPα.

In some embodiments, the agent is selected from the group consisting of a viral vector construct expressing CD47, an adeno-associated virus (AAV) expression cassette comprising a nucleic acid molecule encoding CD47; a small molecule activator of CD47; an anti-CD47 agonistic antibody or antigen-binding fragment thereof; a CD47 fusion protein; a CD47 protein or fragment thereof; an AAV expression cassette comprising a nucleic acid molecule encoding SIRPα; a small molecule activator of SIRPα; an anti-SIRPα agonistic antibody or antigen-binding fragment thereof; a SIRPα fusion protein; and a SIRPα protein or fragment thereof.

In some embodiments, the agent is an AAV expression cassette comprising a nucleic acid molecule encoding CD47.

In some embodiments, the nucleic acid molecule encoding CD47 comprises a nucleotide sequence of any one of SEQ ID NOs:1-8; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of any one of SEQ ID NOs:1-8.

In some embodiments, the nucleic acid molecule encoding CD47 comprises a nucleotide sequence of SEQ ID NO:7; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:7.

In some embodiments, the AAV expression cassette comprises a promoter.

In some embodiments, the promoter is selected from the group consisting of a human red opsin (hRedO) promoter, a human Best1 promoter, a CMV promoter, a CAG promoter and a human Rhodopsin promoter.

In some embodiments, the human red opsin (hRedO) promoter comprises the nucleotide sequence of SEQ ID NO: 9, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:9.

In some embodiments, the human Best 1 promoter comprises the nucleotide sequence of SEQ ID NO: 10, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 10.

In some embodiments, the CMV promoter comprises the nucleotide sequence of SEQ ID NO: 11, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 11.

In some embodiments, the CAG promoter comprises the nucleotide sequence of SEQ ID NO: 12, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 12.

In some embodiments, the human Rhodopsin promoter comprises the nucleotide sequence of SEQ ID NO: 13, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 13.

In some embodiments, the AAV expression cassette further comprises a Kozak sequence. In some embodiments, the Kozak sequence comprises the nucleotide sequence of GCCGCCACC, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of GCCGCCACC.

In some embodiments, the AAV expression cassette further comprises a Woodchuck hepatitis virus posttranscriptional regulatory element (WPRE). In some embodiments, the WPRE comprises the nucleotide sequence of SEQ ID NO: 15, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 15.

In some embodiments, the AAV expression cassette further comprises a polyadenylation signal.

In some embodiments, the polyadenylation signal is a bovine growth hormone polyadenylation signal. In some embodiments, the bovine growth hormone polyadenylation signal comprises the nucleotide sequence of SEQ ID NO: 16, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 16.

In some embodiments, the expression cassette is present in a vector. In some embodiments, the vector is an AAV vector selected from the group consisting of AAV2, AAV 8, AAV2/5, and AAV 2/8.

In one aspect, the present invention provides a method for treating or preventing retinitis pigmentosa in a subject. The method comprises administering to the subject a therapeutically effective amount of an agent that enhances CD47-SIRPα signaling, thereby treating or preventing retinitis pigmentosa in said subject.

In another aspect, the present invention provides a composition comprising an adeno-associated virus (AAV) expression cassette, the expression cassette comprising a promoter and a nucleic acid molecule encoding CD47.

In some embodiments, the nucleic acid molecule encoding CD47 comprises a nucleotide sequence of any one of SEQ ID NOs:1-8; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of any one of SEQ ID NOs:1-8.

In some embodiments, the nucleic acid molecule encoding CD47 comprises a nucleotide sequence of SEQ ID NO: 7; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 7.

In some embodiments, the promoter is selected from a group consisting of a human red opsin (hRedO) promoter, a human Best1 promoter, a CMV promoter, a CAG promoter and a human Rhodopsin promoter.

In some embodiments, the human red opsin (hRedO) promoter comprises the nucleotide sequence of SEQ ID NO: 9, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:9.

In some embodiments, the human Best 1 promoter comprises the nucleotide sequence of SEQ ID NO: 10, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 10.

In some embodiments, the CMV promoter comprises the nucleotide sequence of SEQ ID NO: 11, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 11.

In some embodiments, the CAG promoter comprises the nucleotide sequence of SEQ ID NO: 12, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 12.

In some embodiments, the human Rhodopsin promoter comprises the nucleotide sequence of SEQ ID NO: 13, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 13.

In some embodiments, the AAV expression cassette further comprises a Kozak sequence. In some embodiments, the Kozak sequence comprises the nucleotide sequence of GCCGCCACC, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of GCCGCCACC.

In some embodiments, the AAV expression cassette further comprises a Woodchuck hepatitis virus posttranscriptional regulatory element (WPRE). In some embodiments, the WPRE comprises the nucleotide sequence of SEQ ID NO: 15, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 15.

In some embodiments, the AAV expression cassette further comprises a polyadenylation signal.

In some embodiments, the polyadenylation signal is a bovine growth hormone polyadenylation signal. In some embodiments, the bovine growth hormone polyadenylation signal comprises the nucleotide sequence of SEQ ID NO: 16, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 16.

In some embodiments, the expression cassette is present in a vector. In some embodiments, the vector is an AAV vector selected from the group consisting of AAV2, AAV 8, AAV2/5, and AAV 2/8.

In one aspect, the present invention provides an AAV vector particle comprising the composition of the present invention.

In another aspect, the present invention provides an isolated cell comprising the AAV particle of the present invention.

In one aspect, the present invention provides a pharmaceutical composition comprising the AAV composition of the present invention or the particle of the present invention.

In some embodiments, the pharmaceutical composition further comprises a viscosity inducing agent.

In some embodiments, the pharmaceutical composition is formulated for intraocular administration.

In some embodiments, the intraocular administration is selected from the group consisting of intravitreal or subretinal, subvitreal, subconjuctival, sub-tenon, periocular, retrobulbar, suprachoroidal, and/or intrascleral administration.

In one aspect, the present invention provides a method for prolonging the viability of a photoreceptor cell compromised by a degenerative ocular disorder. The method comprises contacting said cell with the composition of the present invention, the AAV viral particle of the present invention, or the pharmaceutical composition of the present invention, thereby prolonging the viability of the photoreceptor cell compromised by the degenerative ocular disorder.

In another aspect, the present invention provides a method for treating or preventing a degenerative ocular disorder in a subject. The method comprises contacting said cell with the composition of the present invention, the AAV viral particle of the present invention, or the pharmaceutical composition of the present invention, thereby treating or preventing said degenerative ocular disorder.

In one aspect, the present invention provides a method for delaying loss of functional vision in a subject having a degenerative ocular disorder. The method comprises contacting said cell with the composition of the present invention, the AAV viral particle of the present invention, or the pharmaceutical composition of the present invention, thereby delaying loss of functional vision in the subject having the degenerative ocular disorder

In another aspect, the present invention provides a method for treating or preventing retinitis pigmentosa in a subject. The method comprises contacting said cell with the composition of the present invention, the AAV viral particle of the present invention, or the pharmaceutical composition of the present invention, thereby treating or preventing retinitis pigmentosa in said subject.

Other features and advantages of the invention will be apparent from the following detailed description and figures.

### BRIEF DESCRIPTION OF THE DRAWINGS

FIGS. 1A-C depict the association of cone degeneration with increased microglial phagocytosis. FIG. 1A depicts the cone arrestin immunostaining and CX3CR1-positive microglia in flat-mounted retinas from sighted CX3CR1^{GFP/+} (rd1 heterozygous) and rd1;CX3CR1^{GFP/+} mice at P20 and P50. Images were acquired from the central retina. Scale bars, 50 μm. FIG. 1B depicts the schematic of ex vivo phagocytosis assay. Retinas from sighted CX3CR1^{GFP/+} and rd1;CX3CR1^{GFP/+} mice were incubated with yeast (zymosan) particles conjugated to pHrodo Red, a pH-sensitive dye that fluoresces upon lysosomal acidification. Microglia were subsequently analyzed by flow cytometry. FIG. 1C depicts the flow cytometry gating for microglia and quantification of microglial phagocytosis in sighted CX3CR1^{GFP/+} and rd1;CX3CR1^{GFP/+} retinas at P20 and P50. Data are shown as mean±SEM. **** P<0.0001 by two-tailed Student's t-test.

FIGS. 2A-D depict the effect of CD47 expression on cone survival. FIG. 2A depicts the schematics of AAV vectors. AAV8-RedO-FLEX-CD47 is a flip-excision (FLEX) vector in which the CD47 transgene is inverted and flanked by lox2272 (black triangles) and loxP (white triangles) sites. FIG. 2B depicts the immunostaining for CD47 in P40 wild-type (CD-1) retinas treated with AAV8-RedO-GFP or AAV8-RedO-GFP plus AAV8-RedO-CD47. Nuclei were labeled with 4′,6-diamidino-2-phenylindole (DAPI). Scale bars, 50 μm. FIG. 2C depicts the representative flat-mounts of P50 rd1 retinas treated with AAV8-RedO-GFP, AAV8-RedO-GFP plus AAV8-RedO-CD47, or AAV8-RedO-GFP plus AAV8-RedO-FLEX-CD47. Paired images depict low and high magnifications. Scale bars, 1 mm. FIG. 2D depicts the quantification of GFP-positive cones in central retinas of rd1 mice (n=11-17) treated with AAV8-RedO-GFP, AAV8-RedO-GFP plus AAV8-RedO-CD47, or AAV8-RedO-GFP plus AAV8-FLEX-RedO-CD47. Data are shown as mean±SEM. **** P<0.0001 by two-tailed Student's t-test.

FIGS. 3A-B depict the quantification of cone survival by flow cytometry. FIG. 3A depicts the flow cytometry gating for GFP-positive cones in rd1 and rd10 retinas treated with AAV8-RedO-GFP or AAV8-RedO-GFP plus AAV8-RedO-CD47. FIG. 3B depicts the quantification by flow cytometry of GFP-positive cones in P50 rd1 (n=16-20) and P100 rd10 (n=10) retinas treated with AAV8-RedO-GFP or AAV8-RedO-GFP plus AAV8-RedO-CD47. Data are shown as mean±SEM. ** P<0.01 by two-tailed Student's t-test.

FIGS. 4A-B depict the quantification of cone survival by immunostaining. FIG. 4A depicts the representative flat-mounts of P50 rd1 retinas without treatment or treated with AAV8-RedO-GFP or AAV8-RedO-GFP plus AAV8-RedO-CD47 after cone arrestin immunostaining. Paired images depict low and high magnifications. Scale bars, 1 mm. FIG. 4B depicts the quantification of cone arrestin immunostaining in central retinas of rd1 mice (n=10-14) without treatment or treated with AAV8-RedO-GFP or AAV8-RedO-GFP plus AAV8-RedO-CD47. Data are shown as mean±SEM. P<0.001, **** P<0.0001 by two-tailed Student's t-test.

FIGS. 5A-E depict the effect of CD47 expression on long-term cone survival and visual function. FIG. 5A depicts the representative flat-mounts of P130 rd10 retinas treated with AAV8-RedO-GFP or AAV8-RedO-GFP plus AAV8-RedO-CD47. Paired images depict low and high magnifications. Scale bars, 1 mm. FIG. 5B depicts the representative flat-mounts of P150 Rho^{-/-} retinas treated with AAV8-RedO-GFP or AAV8-RedO-GFP plus AAV8-RedO-CD47. Paired images depict low and high magnifications. Scale bars, 1 mm. FIG. 5C depicts the quantification of GFP-positive cones in central retinas of rd10 (n=20) and Rho^{-/-} (n=18) mice treated with AAV8-RedO-GFP or AAV8-RedO-GFP plus AAV8-RedO-CD47. Data are shown as mean±SEM. ** P<0.01, **** P<0.0001 by two-tailed Student's t-test. FIG. 5D depicts the percent time spent in dark in a 50:50 light-dark box for untreated (n=7-10) and rd1 (n=11-13) mice treated with AAV8-RedO-GFP or AAV8-RedO-GFP plus AAV8-RedO-CD47. Data are shown as mean±SEM. *** P<0.001 by two-tailed Student's t-test with Bonferroni correction. ns, not significant. FIG. 5E depicts the visual acuity in eyes from P60 rd10 mice (n=19) as measured by optomotor after treatment with AAV8-RedO-GFP or AAV8-RedO-GFP plus AAV8-RedO-CD47. Data are shown as mean±SEM. ** P<0.0001 by two-tailed Student's t-test.

FIGS. 6A-D depict the effect of delayed CD47 expression on cone survival. FIG. 6A depicts the schematic of delayed AAV expression experiments. PO-1 rd1;CreERT2/+ mice were subretinally injected with FLEX vectors, which were subsequently activated by intraperitoneal (IP) injections of tamoxifen from P19-21. FIG. 6B depicts the Representative flat-mounts of P30 rd1;CreERT2/+ retinas treated with AAV8-RedO-FLEX-mCherry with or without IP tamoxifen. Boundaries of each retina are depicted in yellow. Scale bars, 1 mm. FIG. 6C depicts the representative images of central retinas from P50 rd1; CreERT2/+ mice treated with AAV8-RedO-GFP plus AAV8-RedO-FLEX-CD47 with or without IP tamoxifen. Scale bars, 500 μm. FIG. 6D depicts the quantification of GFP-positive cones in central retinas of rd1; CreERT2/+ mice (n=10-12) treated with AAV8-RedO-GFP plus AAV8-RedO-FLEX-CD47 with or without IP tamoxifen. Data are shown as mean±SEM. **** P<0.0001 by two-tailed Student's t-test.

FIGS. 7A-D depict the role of microglia in CD47-mediated cone survival. FIG. 7A depicts the flow cytometry gating for retinal microglia from P35 rd1 mice with or without PLX5622 from P20-34. Microglia were defined as CD11b-positive Ly6G/Ly6C-negative cells. FIG. 7B depicts the quantification by flow cytometry of retinal microglia from P35 rd1 mice (n=2-4) treated with AAV8-RedO-GFP or AAV8-RedO-GFP plus AAV8-RedO-CD47 with or without PLX5622 from P20-34. Data are shown as mean±SEM. *** P<0.001 by two-tailed Student's t-test. FIG. 7C depicts the representative images of central retinas from P50 rd1 mice treated with AAV8-RedO-GFP or AAV8-RedO-GFP plus AAV8-RedO-CD47 and PLX5622 from P20-49. Scale bars, 500 μm. FIG. 7D depicts the quantification of GFP-positive cones in central retinas of rd1 mice (n=16-18) treated with AAV8-RedO-GFP or AAV8-RedO-GFP plus AAV8-RedO-CD47 and PLX5622 from P20-49. Groups without PLX5622 are taken from FIG. 2D. ns, not significant.

FIGS. 8A-C depict the role of TSP1 and SIRPα in CD47-mediated cone survival. FIG. 8A depicts the immunostaining for TSP1 and SIRPα, respectively, in P30 rd1;TSP1^{-/-} and rd1;SIRPα^{-/-} retinas or heterozygous controls. Scale bars, 50 μm. FIG. 8B depicts the representative images of central retinas from P50 rd1;TSP1^{-/-} and rd1;SIRPα^{-/-} mice treated with AAV8-RedO-GFP or AAV8-RedO-GFP plus AAV8-RedO-CD47. Scale bars, 500 μm. FIG. 8C depicts the quantification of GFP-positive cones in central retinas of rd1;TSP1^{-/-} (n=12) and rd1;SIRPα^{-/-} (n=14-17) mice treated with AAV8-RedO-GFP or AAV8-RedO-GFP plus AAV8-RedO-CD47. Data are shown as mean±SEM. **** P<0.0001 by two-tailed Student's t-test. ns, not significant.

FIG. 9 depicts an exemplary vector map of an exemplary AAV vector of the invention comprising a human RedO promoter and a nucleic acid molecule encoding CD47.

### DETAILED DESCRIPTION OF THE INVENTION

The present invention is based, at least in part on the discovery of mutation-independent compositions and methods of treatment for subjects having RP. More specifically, it has surprisingly been discovered that augmentation of CD47-SIRPα signaling could promote cone survival in RP-mutant mice. The inventors have successfully developed an adeno-associated viral (AAV) vector expressing CD47 (AAV8-RedO-CD47), and have surprisingly discovered that delivery of this vector expressing CD47 in cones promoted cone survival and helped preserve visual function in multiple RP-mutant mice, and that this protection mediated by CD47 requires SIRPα.

It has also surprisingly been discovered that delayed expression of CD47 could also preserve cones, indicating that this therapy would be beneficial even if administered later in disease, after most rods have died. Therefore, augmentation of CD47-SIRPα signaling by, for example, delivery of AAV8-RedO-CD47, offers a potential treatment option for many patients with RP, including those who are diagnosed at older age or with genetics that preclude straightforward gene replacement. This approach may further help combat cone death in other inherited retinal diseases and degenerative retinal disorders. Moreover, augmentation of CD47-SIRPα signaling could also serve as a treatment strategy for other forms of neurodegeneration disorders, such as age-related macular degeneration (AMD), multiple sclerosis, glaucoma or amyotrophic lateral sclerosis (ALS).

Accordingly, the present invention provides compositions, e.g., pharmaceutical compositions, which include an agent that enhances CD47-SIRPα signaling, e.g., a recombinant adeno-associated virus (AAV) vector expressing CD47, and methods of treating a subject having a degenerative ocular disorder, e.g., retinitis pigmentosa.

Various aspects of the invention are described in further detail in the following subsections:

### I. Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. A nucleic acid molecule used in the methods of the present invention can be isolated using standard molecular biology techniques. Using all or portion of a nucleic acid sequence of interest as a hybridization probe, nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

An “isolated” nucleic acid molecule is one which is separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid molecule) in the genomic DNA of the organism from which the nucleic acid molecule is derived.

A nucleic acid molecule for use in the methods of the invention can also be isolated by the polymerase chain reaction (PCR) using synthetic oligonucleotide primers designed based upon the sequence of a nucleic acid molecule of interest. A nucleic acid molecule used in the methods of the invention can be amplified using cDNA, mRNA or, alternatively, genomic DNA as a template and appropriate oligonucleotide primers according to standard PCR amplification techniques. Furthermore, oligonucleotides corresponding to nucleotide sequences of interest can be prepared by standard synthetic techniques, e.g., using an automated DNA synthesizer.

The nucleic acids for use in the methods of the invention can also be prepared, e.g., by standard recombinant DNA techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes or nucleic acid molecules to which they are operatively linked and are referred to as “expression vectors” or “recombinant expression vectors.”. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In some embodiments, “expression vectors” are used in order to permit pseudotyping of the viral envelope proteins.

Expression vectors are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses, adeno-associated viruses, lentiviruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells, those which are constitutively active, those which are inducible, and those which direct expression of the nucleotide sequence only in certain host cells (e.g., tissue-specific regulatory sequences). The expression vectors of the invention can be introduced into host cells to thereby produce proteins or portions thereof, including fusion proteins or portions thereof, encoded by nucleic acids as described herein.

The terms “transformation,” “transfection,” and “transduction” refer to introduction of a nucleic acid, e.g., a viral vector, into a recipient cell.

As used herein, the term “subject” includes warm-blooded animals, preferably mammals, including humans. In a preferred embodiment, the subject is a primate. In an even more preferred embodiment, the primate is a human.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (i.e., contacting a cell with an agent) is intended to include incubating the agent and the cell together in vitro (e.g., adding the agent to cells in culture) or administering the agent to a subject such that the agent and cells of the subject are contacted in vivo. The term “contacting” is not intended to include exposure of cells to an agent that may occur naturally in a subject (i.e., exposure that may occur as a result of a natural physiological process).

As used herein, the term “administering” to a subject includes dispensing, delivering or applying a composition of the invention to a subject by any suitable route for delivery of the composition to the desired location in the subject, including delivery by intraocular administration or intravenous administration. Alternatively or in combination, delivery is by the topical, parenteral or oral route, intracerebral injection, intramuscular injection, subcutaneous/intradermal injection, intravenous injection, buccal administration, transdermal delivery and administration by the rectal, colonic, vaginal, intranasal or respiratory tract route.

As used herein, the term “degenerative ocular disorder” refers generally to a disorder of the retina. In one embodiment, the degenerative ocular disorder is associated with death, of cone cells, and/or rod cells. Moreover, in a particular embodiment, a degenerative ocular disorder is not associated with blood vessel leakage and/or growth, for example, as is the case with diabetic retinopathy, but, instead is characterized primarily by reduced viability of cone cells and/or rod cells. In certain embodiments, the degenerative ocular disorder is a genetic or inherited disorder. In a particular embodiment, the degenerative ocular disorder is retinitis pigmentosa. In another embodiment, the degenerative ocular disorder is age-related macular degeneration. In another embodiment, the degenerative ocular disorder is cone-rod dystrophy. In another embodiment, the degenerative ocular disorder is rod-cone dystrophy. In other embodiments, the degenerative ocular disorder is not associated with blood vessel leakage and/or growth. In certain embodiments, the degenerative ocular disorder is not associated with diabetes and/or diabetic retinopathy. In further embodiments, the degenerative ocular disorder is not NARP (neuropathy, ataxia, and retinitis pigmentosa). In yet further embodiments, the degenerative ocular disorder is not a neurological disorder. In certain embodiments, the degenerative ocular disorder is not a disorder that is associated with a compromised optic nerve and/or disorders of the brain. In the foregoing embodiments, the degenerative ocular disorder is associated with a compromised photoreceptor cell, and is not a neurological disorder.

As used herein, the term “retinitis pigmentosa” or “RP” is known in the art and encompasses a disparate group of genetic disorders of rods and cones. Retinitis pigmentosa generally refers to retinal degeneration often characterized by the following manifestations: night blindness, progressive loss of peripheral vision, eventually leading to total blindness; ophthalmoscopic changes consist in dark mosaic-like retinal pigmentation, attenuation of the retinal vessels, waxy pallor of the optic disc, and in the advanced forms, macular degeneration. In some cases there can be a lack of pigmentation. Retinitis pigmentosa can be associated to degenerative opacity of the vitreous body, and cataract. Family history is prominent in retinitis pigmentosa; the pattern of inheritance may be autosomal recessive, autosomal dominant, or X-linked; the autosomal recessive form is the most common and can occur sporadically.

As used herein, the terms “Cone-Rod Dystrophy” or “CRD” and “Rod-Cone Dystrophy” or “RCD” refer to art recognized inherited progressive diseases that cause deterioration of the cone and rod photoreceptor cells and often result in blindness. CRD is characterized by reduced viability or death of cone cells followed by reduced viability or death of rod cells. By contrast, RCD is characterized by reduced viability or death of rod cells followed by reduced viability or death of cone cells.

As used herein, the term “age-related macular degeneration” also referred to as “macular degeneration” or “AMD”, refers to the art recognized pathological condition which causes blindness amongst elderly individuals. Age related macular degeneration includes both wet and dry forms of AMD. The dry form of AMD, which accounts for about 90 percent of all cases, is also known as atrophic, nonexudative, or drusenoid (age-related) macular degeneration. With the dry form of AMD, drusen typically accumulate in the retinal pigment epithelium (RPE) tissue beneath/within the Bruch's membrane. Vision loss can then occur when drusen interfere with the function of photoreceptors in the macula. The dry form of AMD results in the gradual loss of vision over many years. The dry form of AMD can lead to the wet form of AMD. The wet form of AMD, also known as exudative or neovascular (age-related) macular degeneration, can progress rapidly and cause severe damage to central vision. The macular dystrophies include Stargardt Disease, also known as Stargardt Macular Dystrophy or Fundus Flavimaculatus, which is the most frequently encountered juvenile onset form of macular dystrophy.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

As used herein, the terms “treating” or “treatment” refer to a beneficial or desired result including, but not limited to, alleviation or amelioration of one or more symptoms, diminishing the extent of infection, stabilized (i.e., not worsening) state of infection, amelioration or palliation of the infectious state, whether detectable or undetectable. “Treatment” can also mean prolonging survival as compared to expected survival in the absence of treatment.

Various additional aspects of the methods of the invention are described in further detail in the following subsections.

### II. Methods of the Invention

The present invention is based, at least in part, on the discovery that augmentation of CD47-SIRPα signaling could promote cone survival in RP-mutant mice. In particular, the inventors have surprisingly discovered that delivery of an agent that enhances CD47-SIRPα signaling, e.g., an AAV vector expressing CD47, promoted cone survival and preserved visual function in multiple RP-mutant mice. Therefore, enhancing CD47-SIRPα signaling offers a potential treatment option for many patients with RP, including those who are diagnosed at older ages or with genetics that preclude straightforward gene replacement. This approach may further help combat cone death in other inherited retinal diseases and degenerative retinal disorders. Moreover, augmentation of CD47-SIRPα signaling could also serve as a treatment strategy for other forms of neurodegeneration disorders, such as age-related macular degeneration (AMD), multiple sclerosis, glaucoma or amyotrophic lateral sclerosis (ALS).

Accordingly, in one aspect, the present invention provides methods for treating or preventing a degenerative ocular disorder, e.g., retinitis pigmentosa, age related macular degeneration, cone rod dystrophy, rod cone dystrophy or glaucoma, in a subject. The methods comprise administering to the subject a therapeutically effective amount of an agent that enhances CD47-SIRPα signaling, thereby treating or preventing the degenerative ocular disorder in the subject.

The present invention further provides methods for preventing loss of functional vision in a subject having a degenerative ocular disorder, e.g., retinitis pigmentosa, age related macular degeneration, cone rod dystrophy, rod cone dystrophy or glaucoma. The methods comprise administering to the subject a therapeutically effective amount of an agent that enhances CD47-SIRPα signaling, thereby preventing loss of functional vision in the subject.

The present invention further provides methods for prolonging the viability of a photoreceptor cell compromised by a degenerative ocular disorder, e.g., retinitis pigmentosa, age related macular degeneration, cone rod dystrophy, rod cone dystrophy or glaucoma. The methods comprise administering to the subject a therapeutically effective amount of an agent that enhances CD47-SIRPα signaling, thereby prolonging the viability of a photoreceptor cell compromised by the degenerative ocular disorder.

In another aspect, the present invention provides methods of treating a subject having retinitis pigmentosa. The methods comprise administering to the subject a therapeutically effective amount of an agent that enhances CD47-SIRPα signaling, thereby treating or preventing retinitis pigmentosa in the subject.

In yet another aspect, the present invention provides methods of preventing degeneration of a cone photoreceptor cell in a subject. The methods comprise administering to the subject a therapeutically effective amount of an agent that enhances CD47-SIRPα signaling, thereby preventing degeneration of the cone photoreceptor cell in the subject.

The agents that enhance CD47-SIRPα signaling suitable for use in the methods of the present invention include any compounds or molecules that can increase the expression and/or activity of CD47 and/or SIRPα, for example, the mRNA expression and/or protein expression of CD47 and/or SIRPα; the mRNA and/or protein stability of CD47 and/or SIRPα; and/or the biological activity of CD47 and/or SIRPα. An agent can increase the expression and/or activity of CD47 and/or SIRPα either directly or indirectly. Exemplary agents suitable for use in the methods of the invention include viral vectors, e.g., adenovirus, adeno-associated virus, lentivirus, or retrovirus vector constructs expressing CD47; adeno-associated virus (AAV) expression cassettes comprising a nucleic acid molecule encoding CD47 and/or SIRPα; small molecule activators of CD47 and/or SIRPα; anti-CD47 and/or anti-SIRPα agonist antibodies or antigen-binding fragments thereof; recombinant fusion proteins, e.g., a CD47 fusion protein, or a SIRPα fusion protein; stimulatory peptides, e.g., a CD47 activating peptide or a SIRPα activating peptide. Agents suitable for use in the methods of the invention are discussed in detail below.

Generally, methods are known in the art for delivery of agents into the cells of interest. The agents can be placed in contact with the cell of interest or alternatively, can be injected into a subject suffering from a degenerative ocular disorder. Guidance in the introduction of the compositions of the invention into subjects for therapeutic purposes are known in the art and may be obtained in the above-referenced publications, as well as in U.S. Pat. Nos. 5,631,236, 5,688,773, 5,691,177, 5,670,488, 5,529,774, 5,601,818, and PCT Publication No. WO 95/06486, the entire contents of which are incorporated herein by reference.

The compositions of the invention may be delivered to a subject by, for example, intravenous injection, local administration (see, e.g., U.S. Pat. No. 5,328,470), stereotactic injection (see, e.g., Chen et al. (1994) Proc. Natl. Acad. Sci. U.S.A. 91:3054-3057), or by in vivo electroporation (see, e.g., Matsuda and Cepko (2007) Proc. Natl. Acad. Sci. U.S.A. 104:1027-1032). Preferably, the compositions of the invention are administered to the subject locally. Local administration of the compositions described herein can be by any suitable method in the art including, for example, injection (e.g., intravitreal or subretinal, subvitreal, subconjuctival, sub-tenon, periocular, retrobulbar, suprachoroidal, and/or intrascleral injection), gene gun, by topical application of the composition in a gel, oil, or cream, by electroporation, using lipid-based transfection reagents, transcleral delivery, by implantation of scleral plugs or a drug delivery device, or by any other suitable transfection method.

Application of the methods of the invention for the treatment and/or prevention of a disorder can result in curing the disorder, decreasing at least one symptom associated with the disorder, either in the long term or short term or simply a transient beneficial effect to the subject.

Accordingly, as used herein, the terms “treat,” “treatment” and “treating” include the application or administration of compositions, as described herein, to a subject who is suffering from a degenerative ocular disease or disorder, or who is susceptible to such conditions with the purpose of curing, healing, alleviating, relieving, altering, remedying, ameliorating, improving or affecting such conditions or at least one symptom of such conditions. As used herein, the condition is also “treated” if recurrence of the condition is reduced, slowed, delayed or prevented.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Therapeutically effective amount,” as used herein, is intended to include the amount of a composition of the invention that, when administered to a patient for treating a degenerative ocular disease, is sufficient to effect treatment of the disease (e.g., by diminishing, ameliorating or maintaining the existing disease or one or more symptoms of disease). The “therapeutically effective amount” may vary depending on the composition, how the composition is administered, the disease and its severity and the history, age, weight, family history, genetic makeup, stage of pathological processes mediated by the disease expression, the types of preceding or concomitant treatments, if any, and other individual characteristics of the patient to be treated.

“Prophylactically effective amount,” as used herein, is intended to include the amount of a composition that, when administered to a subject who does not yet experience or display symptoms of e.g., a degenerative ocular disorder, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the composition, how the composition is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of a composition that produces some desired local or systemic effect at a reasonable benefit/risk ratio applicable to any treatment. A composition employed in the methods of the present invention may be administered in a sufficient amount to produce a reasonable benefit/risk ratio applicable to such treatment.

Subjects suitable for treatment using the regimens of the present invention should have or are susceptible to developing a degenerative disease or disorder, e.g., a degenerative ocular disease or disorder. For example, subjects may be genetically predisposed to development of the disorders. Alternatively, abnormal progression of the following factors including, but not limited to visual acuity, the rate of death of cone and/or rod cells, night vision, peripheral vision, attenuation of the retinal vessels, and other ophthalmoscopic factors associated with degenerative ocular disorders such as retinitis pigmentosa may indicate the existence of or a predisposition to a retinal disorder.

In one embodiment, the disorder includes, but not limited to, retinitis pigmentosa, age related macular degeneration, cone rod dystrophy, rod cone dystrophy and glaucoma. In other embodiments, the disorder is not associated with blood vessel leakage and/or growth. In certain embodiments, the disorder is not associated with diabetes. In another embodiment, the disorder is not diabetic retinopathy. In further embodiments, the disorder is not NARP (neuropathy, ataxia and retinitis pigmentosa). In one embodiment, the disorder is a disorder associated with decreased viability of cone and/or rod cells. In yet another embodiment, the disorder is a genetic disorder. In some embodiments, the disorder is a neurodegeneration disorder, such as age-related macular degeneration (AMD), multiple sclerosis, glaucoma or amyotrophic lateral sclerosis (ALS).

The compositions, as described herein, may be administered as necessary to achieve the desired effect and depend on a variety of factors including, but not limited to, the severity of the condition, age and history of the subject and the nature of the composition, for example, the identity of the genes or the affected biochemical pathway.

The pharmaceutical compositions of the invention may be administered in a single dose or, in particular embodiments of the invention, multiples doses (e.g. two, three, four, or more administrations) may be employed to achieve a therapeutic effect.

The therapeutic or preventative regimens may cover a period of at least about 2, 3, 4, 5, 6, 7, 8, 9, 10, 11, 12, 13, 14, 15, 16, 17, 18, 19, 20, 21, 22, 23 or 24 weeks, or be chronically administered to the subject.

In one embodiment, the viability or survival of photoreceptor cells, such as cones cells, is, e.g., about 1 week, about 2 weeks, about 3 weeks, about 4 weeks, about 5 weeks, about 6 weeks, about 7 weeks, about 8 weeks, about 3 years, about 4 years, about 5 years, about 10 years, about 15, years, about 20 years, about 25 years, about 30 years, about 40 years, about 50 years, about 60 years, about 70 years, and about 80 years.

In general, the compositions of the invention, comprising agents that enhance CD47 and/or SIRPα signaling, are provided in a therapeutically effective amount to elicit the desired effect, e.g., increase CD47 and/or SIRPα expression and/or activity. The quantity of the compositions to be administered, both according to number of treatments and amount, will also depend on factors such as the clinical status, age, previous treatments, the general health and/or age of the subject, other diseases present, and the severity of the disorder. Precise amounts of active ingredient required to be administered depend on the judgment of the gene therapist and will be particular to each individual patient. Moreover, treatment of a subject with a therapeutically effective amount of the compositions of the invention can include a single treatment or, preferably, can include a series of treatments. It will also be appreciated that the effective dosage used for treatment may increase or decrease over the course of a particular treatment. Changes in dosage may result from the results of diagnostic assays as described herein. The pharmaceutical compositions can be included in a container, pack, or dispenser together with instructions for administration.

In certain embodiments, the agent that enhances CD47 and/or SIRPα signaling is an adeno-associated virus (AAV) expression cassette comprising a nucleic acid molecule encoding CD47, or a AAV vector particle comprising the nucleic acid molecules and/or the vectors of the invention.

In some embodiments, a therapeutically effective amount or a prophylactically effective amount of a viral particle of the invention (or pharmaceutical composition of the invention) is in titers ranging from about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹² viral particles (vp).

In some embodiments, a therapeutically effective amount or a prophylactically effective amount of a viral particle of the invention (or pharmaceutical composition of the invention) is in genome copies (“GC”), also referred to as “viral genomes” (“vg”) ranging from about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹² vg.

Any method known in the art can be used to determine the genome copy (GC) number of the viral compositions of the invention. One method for performing AAV GC number titration is as follows: purified AAV viral particle samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In various embodiments, the methods of the present invention further comprises monitoring the effectiveness of treatment. For example, visual acuity, the rate of death of cone and/or rod cells, night vision, peripheral vision, attenuation of the retinal vessels, and other ophthalmoscopic changes associated with retinal disorders such as retinitis pigmentosa may be monitored to assess the effectiveness of treatment. Additionally, the rate of death of cells associated with the particular disorder that is the subject of treatment and/or prevention, may be monitored. Alternatively, the viability of such cells may be monitored, for example, as measured by phospholipid production. Electroretinography (ERG) can also be used to monitor the effectiveness of treatment (e.g., electroretinography—ERG). Furthermore, the effectiveness of treatment can be monitored by behavior assays, such as light-dark discrimination tests or optomotor assays, as described in the Examples.

In certain embodiments of the invention, a composition of the invention is administered in combination with an additional therapeutic agent or treatment. The compositions and an additional therapeutic agent can be administered in combination in the same composition or the additional therapeutic agent can be administered as part of a separate composition or by another method described herein.

Examples of additional therapeutic agents suitable for use in the methods of the invention include those agents known to treat retinal disorders, such as retinitis pigmentosa and age-related macular degeneration and include, for example, fat soluble vitamins (e.g., vitamin A, vitamin E, and ascorbic acid), calcium channel blockers (e.g., diltiazem) carbonic anhydrase inhibitors (e.g., acetazolamide and methazolamide), anti-angiogenics (e.g., antiVEGF antibodies), growth factors (e.g., rod-derived cone viability factor (RdCVF), BDNF, CNTF, bFGF, and PEDF), antioxidants, other gene therapy agents (e.g., optogenetic gene therapy, e.g., channelrhodopsin, melanopsin, and halorhodopsin), and compounds that drive photoreceptor regeneration by, e.g., reprogramming Müller cells into photoreceptor progenitors (e.g., alpha-aminoadipate). Exemplary treatments for use in combination with the treatment methods of the present invention include, for example, retinal and/or retinal pigmented epithelium transplantation, stem cell therapies, retinal prostheses, laser photocoagulation, photodynamic therapy, low vision aid implantation, submacular surgery, and retinal translocation.

### III. Compositions of the Invention

As described above, enhancing CD47-SIRPα signaling could promote cone survival in multiple RP-mutant mice and help preserve visual function. Accordingly, agents which enhance CD47-SIRPα signaling, e.g., increase the expression and/or activity of CD47 and/or SIRPα, are useful in the methods of the present invention. Exemplary agents can be a nucleic acid, a polypeptide, an antibody, or a small molecule compound. These stimulatory or activating agents may function at a level of transcription, mRNA stability, translation, protein stability, protein degradation, protein modification, or protein binding.

### A. AAV Expression Cassettes and Vectors

In some embodiments, the agents that enhance CD47-SIRPα signaling are adeno-associated viral (AAV) expression cassettes, AAV expression cassettes present in AAV vectors, or AAV vectors comprising a recombinant viral genome which include an expression cassette.

Accordingly, in one aspect the present invention provides compositions comprising an adeno-associated virus (AAV) expression cassette, the expression cassette comprising a promoter and a nucleic acid molecule encoding CD47 molecule (CD47) and/or signal regulatory protein alpha (SIRPα).

In one aspect, the present invention provides compositions comprising an adeno-associated virus (AAV) expression cassette, the expression cassette comprising a promoter and a nucleic acid molecule encoding CD47.

In another aspect, the present invention provides compositions comprising an adeno-associated virus (AAV) expression cassette, the expression cassette comprising a promoter and a nucleic acid molecule encoding SIRPα.

The terms “adeno-associated virus”, “AAV virus”, “AAV virion”, “AAV viral particle”, and “AAV particle”, as used interchangeably herein, refer to a viral particle composed of at least one AAV capsid protein (preferably by all of the capsid proteins of a particular AAV serotype) and an encapsidated polynucleotide AAV genome. If the particle comprises a heterologous polynucleotide (i.e. a polynucleotide other than a wild-type AAV genome such as a transgene to be delivered to a mammalian cell) flanked by the AAV inverted terminal repeats (ITRs), it is typically referred to as an “AAV vector particle.”

AAV viruses belonging to the genus Dependovirus of the Parvoviridae family and, as used herein, include any serotype of the over 100 serotypes of AAV viruses known. In general, serotypes of AAV viruses have genomic sequences with a significant homology at the level of amino acids and nucleic acids, provide an identical series of genetic functions, produce virions that are essentially equivalent in physical and functional terms, and replicate and assemble through practically identical mechanisms.

The AAV genome is approximately 4.7 kilobases long and is composed of single-stranded deoxyribonucleic acid (ssDNA) which may be either positive- or negative-sensed. The genome comprises inverted terminal repeats (ITRs) at both ends of the DNA strand, and two open reading frames (ORFs): rep and cap. The rep frame is made of four overlapping genes encoding Rep proteins required for the AAV life cycle. The cap frame contains overlapping nucleotide sequences of capsid proteins: VP1, VP2 and VP3, which interact together to form a capsid of an icosahedral symmetry. See Carter B, Adeno-associated virus and adeno-associated virus vectors for gene delivery, Lassie D, et ah, Eds., “Gene Therapy: Therapeutic Mechanisms and Strategies” (Marcel Dekker, Inc., New York, NY, US, 2000) and Gao G, et al, J. Virol. 2004; 78(12):6381-6388.

The term “AAV vector” or “AAV construct” refers to a vector derived from an adeno-associated virus serotype, including without limitation, AAV-1, AAV-2, AAV-3, AAV-4, AAV-5, AAV6, AAV7, AAV8, and AAV9. “AAV vector” refers to a vector that includes AAV nucleotide sequences as well as heterologous nucleotide sequences. AAV vectors require only the 145 base terminal repeats in cis to generate virus. All other viral sequences are dispensable and may be supplied in trans (Muzyczka (1992) Curr. Topics Microbiol. Immunol. 158:97-129). Typically, the rAAV vector genome will only retain the inverted terminal repeat (ITR) sequences so as to maximize the size of the transgene that can be efficiently packaged by the vector. The ITRs need not be the wild-type nucleotide sequences, and may be altered, e.g., by the insertion, deletion or substitution of nucleotides, as long as the sequences provide for functional rescue, replication and packaging.

In particular embodiments, the AAV vector is an AAV2, AAV2.7m8, AAV2/5 or AAV2/8 vector. Suitable AAV vectors are described in, for example, U.S. Pat. No. 7,056,502 and Yan et al. (2002) J. Virology 76(5):2043-2053, the entire contents of which are incorporated herein by reference.

Such AAV vectors can be replicated and packaged into infectious viral particles when present in a host cell that has been transfected with a vector encoding and expressing rep and cap gene products (i.e. AAV Rep and Cap proteins), and wherein the host cell has been transfected with a vector which encodes and expresses a protein from the adenovirus open reading frame E4orf6.

The term “cap gene” or “AAV cap gene”, as used herein, refers to a gene that encodes a Cap protein. The term “Cap protein”, as used herein, refers to a polypeptide having at least one functional activity of a native AAV Cap protein (e.g. VP1, VP2, VP3). Examples of functional activities of Cap proteins (e.g. VP1, VP2, VP3) include the ability to induce formation of a capsid, facilitate accumulation of single-stranded DNA, facilitate AAV DNA packaging into capsids (i.e. encapsidation), bind to cellular receptors, and facilitate entry of the virion into host.

The term “capsid”, as used herein, refers to the structure in which the viral genome is packaged. A capsid consists of several oligomeric structural subunits made of proteins. For instance, AAV have an icosahedral capsid formed by the interaction of three capsid proteins: VP1, VP2 and VP3.

The term “genes providing helper functions”, as used herein, refers to genes encoding polypeptides which perform functions upon which AAV is dependent for replication (i.e. “helper functions”). The helper functions include those functions required for AAV replication including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, UL8, UL52, and UL29, and herpesvirus polymerase. In one embodiment, a helper function does not include adenovirus E1.

The term “rep gene” or “AAV rep gene”, as used herein, refers to a gene that encodes a Rep protein. The term “Rep protein”, as used herein, refers to a polypeptide having at least one functional activity of a native AAV Rep protein (e.g. Rep 40, 52, 68, 78). A “functional activity” of a Rep protein (e.g. Rep 40, 52, 68, 78) is any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome.

The term “adeno-associated virus ITRs” or “AAV ITRs”, as used herein, refers to the inverted terminal repeats present at both ends of the DNA strand of the genome of an adeno-associated virus. The ITR sequences are required for efficient multiplication of the AAV genome. Another property of these sequences is their ability to form a hairpin. This characteristic contributes to its self-priming which allows the primase-independent synthesis of the second DNA strand. The ITRs have also shown to be required for efficient encapsidation of the AAV DNA combined with generation of fully assembled, deoxyribonuclease-resistant AAV particles.

The term “expression cassette”, as used herein, refers to a nucleic acid construct, generated recombinantly or synthetically, with a series of specified nucleic acid elements, which permit transcription of a particular nucleic acid in a target cell.

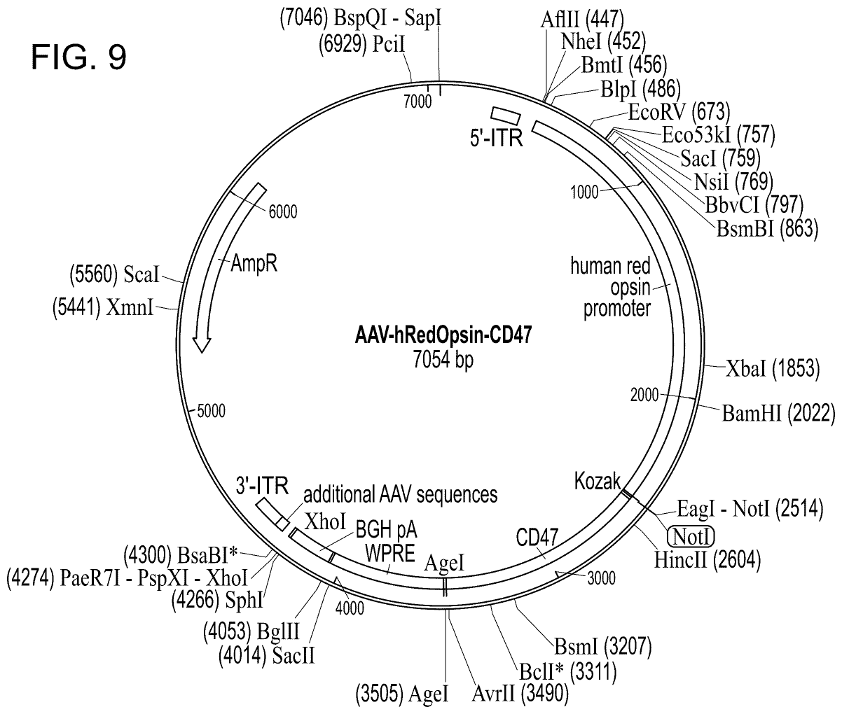

The expression cassettes of the invention include a promoter operably linked to a nucleic acid molecule encoding CD47. Exemplary expression cassettes of the invention are depicted in FIG. 9.

The term “promoter” as used herein refers to a recognition site of a DNA strand to which the RNA polymerase binds. The promoter forms an initiation complex with RNA polymerase to initiate and drive transcriptional activity. The complex can be modified by activating sequences termed “enhancers” or inhibitory sequences termed “silencers”.

Suitable promoters for use in the expression cassettes of the invention may be ubiquitous promoters, such as a CMV promoter, a CAG promoter or an SV40 promoter, and tissue-specific promoters, i.e., promoters that direct expression of a nucleic acid molecule preferentially in a particular cell type.

In one embodiment, the CMV promoter comprises the nucleotide sequence of SEQ ID NO:11, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:11.

In one embodiment, the CAG promoter comprises the nucleotide sequence of SEQ ID NO:12, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:12.

In one embodiment, a tissue-specific promoter for use in the present invention is a photoreceptor-specific (PR-specific) promoter. The PR-specific promoter may be a rod-specific promoter; a cone-specific promoter; or a rod- and cone-specific promoter. In one embodiment, a tissue-specific promoter for use in the present invention is a cone-specific promoter.

Suitable PR-specific promoters are known in the art and include, for example, a human red opsin, a human rhodopsin promoter, a guanine nucleotide-binding protein G subunit alpha-2 (GNAT2) promoter, a human rhodopsin kinase (RK) promoter, a G protein-coupled receptor kinase 1 (GRK1) promoter.

In certain embodiments, a suitable PR-specific promoter is a human red opsin (RedO) promoter.

As used interchangeably herein, the terms “human RO,” “red opsin,” “RedO,” “RO,” and “hRO” refer to Opsin 1, Long Wave Sensitive, also known as Red Cone Photoreceptor Pigment, Opsin 1 (Cone Pigments), Long-Wave-Sensitive, Cone Dystrophy 5 (X-Linked), Red-Sensitive Opsin, RCP, ROP, Long-Wave-Sensitive Opsin, Color Blindness, Protan, Red Cone Opsin, COD5, CBBm, and CBP.

In one embodiment, the RedO promoter comprises the nucleotide sequence of SEQ ID NO:9, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:9.

In certain embodiments, a suitable PR-specific promoter is a human rhodopsin promoter. In one embodiment, the rhodopsin promoter comprises the nucleotide sequence of SEQ ID NO:13, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:13.

In certain embodiments, a tissue-specific promoter for use in the present invention is a retinal pigment epithelium-specific (RPE-specific) promoter. The RPE-specific promoter may be a human Best1 promoter. In one embodiment, the Best1 promoter comprises the nucleotide sequence of SEQ ID NO:10, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:10.

As used herein, the term “CD47” refers to CD47 molecule, a membrane protein, which is involved in the increase in intracellular calcium concentration that occurs upon cell adhesion to extracellular matrix. The encoded protein is also a receptor for the C-terminal cell binding domain of thrombospondin, and it may play a role in membrane transport and signal transduction. CD47 is also known as OA3, MER6, IAP, antigenic surface determinant protein OA3; leukocyte surface antigen CD47; CD47 antigen (Rh-related antigen, integrin-associated signal transducer); antigen identified by monoclonal antibody 1D8; integrin associated protein; Rh-related antigen; CD47 glycoprotein; integrin-associated signal transducer; integrin-associated protein; protein MER6; and CD47 antigen.

The nucleotide and amino acid sequences of human CD47 are known and may be found in, for example, GenBank Reference Sequences NM_001777.4, NM_198793.3, and NM_001382306.1 (SEQ ID NOs:21-23, respectively, the entire contents of each of which are incorporated herein by reference). The coding sequence for human CD47 can be found in SEQ ID Nos: 1-3.

The nucleotide and amino acid sequences of mouse CD47 are known and may be found in, for example, GenBank Reference Sequences NM_001368415.1; NM_001368416.1; NM_001368417.1; NM_010581.3; and NM_001368418.1 (SEQ ID NOs:24-28, respectively, the entire contents of each of which are incorporated herein by reference). The coding sequence for mouse CD47 can be found in SEQ ID Nos: 4-8.

In one embodiment, a nucleic acid molecule encoding CD47 comprises a nucleotide sequence of any one of SEQ ID Nos: 1-8, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of any one of SEQ ID Nos: 1-8.

In one embodiment, a nucleic acid molecule encoding CD47 comprises a nucleotide sequence of SEQ ID No: 4, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 4.

The invention further encompasses nucleic acid molecules that differ, due to degeneracy of the genetic code, from the nucleotide sequence of nucleic acids encoding a CD47 polypeptide, and, thus, encode the same protein.

As used herein, the term “SIRPα” or “signal regulatory protein alpha,” also known as SHPS-1, CD172a, p84, MyD-1, Bit, or PTPNS1, refers to an inhibitory receptor present on myeloid cells that interacts with CD47. Upon binding CD47, SIRPα initiates a signaling cascade that results in the inhibition of phagocytosis.

The nucleotide and amino acid sequences for human SIRPα are known and may be found in, for example, GenBank Reference Sequences NM_001040022.1, NM_001040023.2, NM_080792.3 and NM_001330728.1 (SEQ ID Nos: 29-32, respectively, the entire contents of each of which are incorporated herein by reference).

In one embodiment, a nucleic acid molecule encoding SIRPα comprises nucleotides 361-1875 of SEQ ID NO: 29, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 361-1875 SEQ ID NO: 29.

In one embodiment, a nucleic acid molecule encoding SIRPα comprises nucleotides 42-1556 of SEQ ID NO: 30, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 42-1556 SEQ ID NO: 30.

In one embodiment, a nucleic acid molecule encoding SIRPα comprises nucleotides 65-1579 of SEQ ID NO: 31, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 65-1579 SEQ ID NO: 31.

In one embodiment, a nucleic acid molecule encoding SIRPα comprises nucleotides 361-1887 of SEQ ID NO: 32, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 361-1887 SEQ ID NO: 32.

The nucleotide and amino acid sequences of mouse SIRPα are known and may be found in, for example, GenBank Reference Sequences NM_007547.4; NM_001177647.2, NM_001291019.1, NM_001291020.1, NM_001291021.1, NM_001291022.1, NM_001355158.1 and NM_001355160.1 (SEQ ID NOs:33-40, respectively, the entire contents of each of which are incorporated herein by reference).

In one embodiment, a nucleic acid molecule encoding SIRPα comprises nucleotides 445-1974 of SEQ ID NO: 33, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 445-1974 SEQ ID NO: 33.

In one embodiment, a nucleic acid molecule encoding SIRPα comprises nucleotides 445-1320 of SEQ ID NO: 34, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 445-1320 SEQ ID NO: 34.

In one embodiment, a nucleic acid molecule encoding SIRPα comprises nucleotides 445-1986 of SEQ ID NO: 35, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 445-1986 of SEQ ID NO: 35.

In one embodiment, a nucleic acid molecule encoding SIRPα comprises nucleotides 247-1788 of SEQ ID NO: 36, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 247-1788 SEQ ID NO: 36.

In one embodiment, a nucleic acid molecule encoding SIRPα comprises nucleotides 445-1332 of SEQ ID NO: 37, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 445-1332 SEQ ID NO: 37.

In one embodiment, a nucleic acid molecule encoding SIRPα comprises nucleotides 445-963 of SEQ ID NO: 38, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 445-963 SEQ ID NO: 38.

In one embodiment, a nucleic acid molecule encoding SIRPα comprises nucleotides 247-1776 of SEQ ID NO: 39, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 247-1776 SEQ ID NO: 39.

In one embodiment, a nucleic acid molecule encoding SIRPα comprises nucleotides 37-1530 of SEQ ID NO: 40, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of nucleotides 37-1530 SEQ ID NO: 40.

The invention further encompasses nucleic acid molecules that differ, due to degeneracy of the genetic code, from the nucleotide sequence of nucleic acids encoding a SIRPα polypeptide, and, thus, encode the same protein.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions total # of positions (e.g., overlapping positions)×100).

The determination of percent identity between two sequences may be accomplished using a mathematical algorithm. A non-limiting example of a mathematical algorithm utilized for the comparison of two sequences is the algorithm of Karlin and Altschul (1990) Proc. Natl. Acad. Sol. USA 87:2264-2268, modified as in Karlin and Altschul (1993) Proc. Natl. Acad Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score 50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res 25:3389-3402, which is able to perform gapped local alignments for the programs BLASTN, BLASTP and BLASTX.

In some embodiments, the expression cassettes of the invention further comprise a Kozak sequence between the promoter and the nucleic acid molecule endoing CD47 and/or SIRPα.

As used herein, a “Kozak sequence” refers to a nucleic acid motif that functions as the protein translation initiation site in most eukaryotic mRNA transcript. In one embodiment, the Kozak sequence comprises the nucleotide sequence of GCCGCCACC, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of GCCGCCACC.

In some embodiments, the expression cassettes of the invention further comprise a post-transcriptional regulatory region.

The term “post-transcriptional regulatory region”, as used herein, refers to any polynucleotide that facilitates the expression, stabilization, or localization of the sequences contained in the cassette or the resulting gene product.

In one embodiment, a post-transcriptional regulatory region suitable for use in the expression cassettes of the invention includes a Woodchuck hepatitis virus post-transcriptional regulatory element.

As used herein, the term “Woodchuck hepatitis virus posttranscriptional regulatory element” or “WPRE,” refers to a DNA sequence that, when transcribed, creates a tertiary structure capable of enhancing the expression of a gene. See Lee Y, et al, Exp. Physiol. 2005; 90(1):33-37 and Donello J, et al, J. Virol. 1998; 72(6):5085-5092. In one embodiment, the WPRE comprises the nucleotide sequence of SEQ ID NO: 15, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 15.

In some embodiments, the expression cassettes of the invention further comprises a polyadenylation signal.

As used herein, a “polyadenylation signal” or “polyA signal,” as used herein refers to a nucleotide sequence that terminates transcription. Suitable polyadenylation signals for use in the AAV vectors of the invention are known in the art and include, for example, a bovine growth hormone polyA signal (BGH pA) or an SV40 polyadenylation signal (SV40 polyA). In one embodiment, a BGH pA comprises the nucleotide sequence of SEQ ID NO: 16, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 16.

In some embodiments, the expression cassettes of the invention further comprise an enhancer.

The term “enhancer”, as used herein, refers to a DNA sequence element to which transcription factors bind to increase gene transcription.

In some embodiments, the expression cassettes of the invention further comprise an intron.

As used herein, an “intron” refers to a non-coding nucleic acid molecule which is removed by RNA splicing during maturation of a final RNA product. In one embodiment, the intron is an SV40 intron. In another embodiment, the intron is a human beta-globin intron.

In another embodiment, the intron is a chimeric intron.

A “chimeric intron” is an artificial (or non-naturally occurring intron that enhances mRNA processing and increases expression levels of a downstream open reading frame.

The AAV vectors of the invention may also include cis-acting 5′ and 3′ inverted terminal repeat (ITR) sequences. In some embodiments, the ITR sequences are about 145 bp in length. In some embodiments, substantially the entire sequences encoding the ITRs are used in the molecule. In other embodiments, the ITRs include modifications. Procedures for modifying these ITR sequences are known in the art. See Brown T, “Gene Cloning” (Chapman & Hall, London, G B, 1995), Watson R, et al, “Recombinant DNA”, 2nd Ed. (Scientific American Books, New York, NY, US, 1992), Alberts B, et al, “Molecular Biology of the Cell” (Garland Publishing Inc., New York, NY, US, 2008), Innis M, et al, Eds., “PCR Protocols. A Guide to Methods and Applications” (Academic Press Inc., San Diego, CA, US, 1990), Erlich H, Ed., “PCR Technology. Principles and Applications for DNA Amplification” (Stockton Press, New York, NY, US, 1989), Sambrook J, et al, “Molecular Cloning. A Laboratory Manual” (Cold Spring Harbor Laboratory Press, Cold Spring Harbor, NY, US, 1989), Bishop T, et al, “Nucleic Acid and Protein Sequence. A Practical Approach” (IRL Press, Oxford, G B, 1987), Reznikoff W, Ed., “Maximizing Gene Expression” (Butterworths Publishers, Stoneham, MA, US, 1987), Davis L, et al, “Basic Methods in Molecular Biology” (Elsevier Science Publishing Co., New York, NY, US, 1986), and Schleef M, Ed., “Plasmid for Therapy and Vaccination” (Wiley-VCH Verlag GmbH, Weinheim, D E, 2001).

The AAV vectors of the invention may include ITR nucleotide sequences derived from any one of the AAV serotypes. In a preferred embodiment, the AAV vector comprises 5′ and 3′ AAV ITRs. In one embodiment, the 5′ and 3′ AAV ITRs derive from AAV2. AAV ITRs for use in the AAV vectors of the invention need not have a wild-type nucleotide sequence (See Kotin, Hum. Gene Ther., 1994, 5:793-801). As long as ITR sequences function as intended for the rescue, replication and packaging of the AAV virion, the ITRs may be altered by the insertion, deletion or substitution of nucleotides or the ITRs may be derived from any of several AAV serotypes or its mutations.

In one embodiment, a 5′ ITR comprises a nucleotide sequence of SEQ ID NO: 17, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 17.

In one embodiment, a 3′ ITR comprises a nucleotide sequence of SEQ ID NO: 18, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 18.

In addition, an AAV vector can contain one or more selectable or screenable marker genes for initially isolating, identifying, or tracking host cells that contain DNA encoding the AAV vector (and/or rep, cap and/helper genes), e.g., antibiotic resistance, as described herein.

As indicated above, the AAV vectors of the invention may be packaged into AAV viral particles for use in the methods, e.g., gene therapy methods, of the invention (discussed below) to produce AAV vector particles using methods known in the art.

Such methods generally include packaging the AAV vectors of the invention into infectious AAV viral particles in a host cell that has been transfected with a vector encoding and expressing rep and cap gene products (i.e. AAV Rep and Cap proteins), and with a vector which encodes and expresses a protein from the adenovirus open reading frame E4orf6.

Suitable AAV Caps may be derived from any serotype. In one embodiment, the capsid is derived from the AAV of the group consisting on AAV1, AAV2, AAV3, AAV4, AAV5, AAV6, AAV7, AAV8 and AAV9. In another embodiment, the AAV of the invention comprises a capsid derived from the AAV7, AAV5 or AAV8 serotypes.

In some embodiments, an AAV Cap for use in the method of the invention can be generated by mutagenesis (i.e. by insertions, deletions, or substitutions) of one of the aforementioned AAV Caps or its encoding nucleic acid. In some embodiments, the AAV Cap is at least 70%, 75%, 80%, 85%, 90%, 95%, 98%, or 99% or more similar to one or more of the aforementioned AAV Caps.

In some embodiments, the AAV Cap is chimeric, comprising domains from two, three, four, or more of the aforementioned AAV Caps. In some embodiments, the AAV Cap is a mosaic of VP1, VP2, and VP3 monomers originating from two or three different AAV or a recombinant AAV. In some embodiments, a rAAV composition comprises more than one of the aforementioned Caps.

Suitable rep may be derived from any AAV serotype. In one embodiment, the rep is derived from any of the serotypes selected from the group consisting of AAV1, AAV2, AAV3, AAV4, AAV5, AAV6, AAV7, AAV8, or AAV9. In another embodiment, the AAV rep is derived from the serotype AAV2.

Suitable helper genes may be derived from any AAV serotype and include adenovirus E4, E2a and VA.

The AAV rep, AAV cap and genes providing helper functions can be introduced into the cell by incorporating the genes into a vector such as, for example, a plasmid, and introducing the vector into a cell. The genes can be incorporated into the same plasmid or into different plasmids. In one, the AAV rep and cap genes are incorporated into one plasmid and the genes providing helper functions are incorporated into another plasmid.

The AAV vectors of the invention and the polynucleotides comprising AAV rep and cap genes and genes providing helper functions may be introduced into a host cell using any suitable method well known in the art. See Ausubel F, et al, Eds., “Short Protocols in Molecular Biology”, 4th Ed. (John Wiley and Sons, Inc., New York, NY, US, 1997), Brown (1995), Watson (1992), Alberts (2008), Innis (1990), Erlich (1989), Sambrook (1989), Bishop (1987), Reznikoff (1987), Davis (1986), and Schleef (2001), supra. Examples of transfection methods include, but are not limited to, co-precipitation with calcium phosphate, DEAE-dextran, polybrene, electroporation, microinjection, liposome-mediated fusion, lipofection, retrovirus infection and biolistic transfection. When the cell lacks the expression of any of the AAV rep and cap genes and genes providing adenoviral helper functions, said genes can be introduced into the cell simultaneously with the AAV vector. Alternatively, the genes can be introduced in the cell before or after the introduction of the AAV vector of the invention.

Methods of culturing packaging cells and exemplary conditions which promote the release of AAV vector particles, such as the producing of a cell lysate, are known in the art. Producer cells are grown for a suitable period of time in order to promote release of viral vectors into the media. Generally, cells may be grown for about 24 hours, about 36 hours, about 48 hours, about 72 hours, about 4 days, about 5 days, about 6 days, about 7 days, about 8 days, about 9 days, up to about 10 days. After about 10 days (or sooner, depending on the culture conditions and the particular producer cell used), the level of production generally decreases significantly. Generally, time of culture is measured from the point of viral production. For example, in the case of AAV, viral production generally begins upon supplying helper virus function in an appropriate producer cell as described herein. Generally, cells are harvested about 48 to about 100, preferably about 48 to about 96, preferably about 72 to about 96, preferably about 68 to about 72 hours after helper virus infection (or after viral production begins).

The AAV vector particles of the invention can be obtained from both: i) the cells transfected with the foregoing and ii) the culture medium of the cells after a period of time post-transfection, preferably 72 hours. Any method for the purification of the AAV vector particles from the cells or the culture medium can be used for obtaining the AAV vector particles of the invention. In a particular embodiment, the AAV vector particles of the invention are purified following an optimized method based on a polyethylene glycol precipitation step and two consecutive cesium chloride (CsCl) or iodixanol density gradient ultracentrifugation. See Ayuso et al., 2014, Zolotukhin S, et al., Gene Ther. 1999; 6; 973-985. Purified AAV vector particles of the invention can be dialyzed against an appropriate formulation buffer such as PBS, filtered and stored at −80° C. Titers of viral genomes can be determined by quantitative PCR following the protocol described for the AAV2 reference standard material using linearized plasmid DNA as standard curve. See Aurnhammer C, et al., Hum Gene Ther Methods, 2012, 23, 18-28, D'Costa S, et al., Mol Ther Methods Clin Dev. 2016, 5, 16019.

In some embodiments, the methods further comprise purification steps, such as treatment of the cell lysate with benzonase, purification of the cell lysate with the use of affinity chromatography and/or ion-exchange chromotography. See Halbert C, et al, Methods Mol. Biol. 2004; 246:201-212, Nass S, et al., Mol Ther Methods Clin Dev. 2018 Jun. 15; 9: 33-46.

AAV Rep and Cap proteins and their sequences, as well as methods for isolating or generating, propagating, and purifying such AAV, and in particular, their capsids, suitable for use in producing AAV are known in the art. See Gao, 2004, supra, Russell D, et al, U.S. Pat. No. 6,156,303, Hildinger M, et al, U.S. Pat. No. 7,056,502, Gao G, et al, U.S. Pat. No. 7,198,951, Zolotukhin S, U.S. Pat. No. 7,220,577, Gao G, et al, U.S. Pat. No. 7,235,393, Gao G, et al, U.S. Pat. No. 7,282,199, Wilson J, et al, U.S. Pat. No. 7,319,002, Gao G, et al, U.S. Pat. No. 7,790,449, Gao G, et al, US 20030138772, Gao G, et al, US 20080075740, Hildinger M, et al, WO 2001/083692, Wilson J, et al, WO 2003/014367, Gao G, et al, WO 2003/042397, Gao G, et al, WO 2003/052052, Wilson J, et al, WO 2005/033321, Vandenberghe L, et al, WO 2006/110689, Vandenberghe L, et al, WO 2007/127264, and Vandenberghe L, et al, WO 2008/027084.

### B. Agonist Antibodies

In some embodiments, the agents that enhance CD47-SIRPα signaling for use in the methods of the present invention are agonist antibodies that can bind CD47 or SIRPα and increase the expression and/or activities of CD47 and/or SIRPα.

Agonist antibodies can be identified, screened for (e.g., using phage display), or characterized for their physical/chemical properties and/or biological activities by various assays known in the art (see, for example, Antibodies: A Laboratory Manual, Second edition, Greenfield, ed., 2014). Binding specificity of an antibody for its antigen can be tested by known methods in the art such as ELISA, Western blot, or surface plasmon resonance.

Antibodies may be produced using recombinant methods and compositions known in the art, e.g., as described in U.S. Pat. No. 4,816,567, incorporated by reference herein. An isolated nucleic acid encoding, for example, an anti-CD47 or anti-SIRPα antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): a vector comprising a nucleic acid that encodes an amino acid sequence comprising the VL of the antibody and an amino acid sequence comprising the VH of the antibody, or a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell).

For recombinant production of an anti-CD47 or anti-SIRPα antibody, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.− CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### C. Small Molecule Activators

Other agents that enhance CD47-SIRPα signaling for use in the methods of the present invention include small molecule activators that bind CD47 or SIRPα, and increase the expression and/or activities of CD47 and/or SIRPα. Such compounds can be either natural products or members of a combinatorial chemistry library, and can be identified using screening assays, as described in detail below.

In some embodiments, the small molecule activators of the present invention may increase the interaction between CD47 and SIRPα or their corresponding interacting proteins. In some embodiments, the small molecule activators are selected to bind domains sharing homology to a domain of the CD47 or SIRPα. For example, a small molecule of the present invention may be directed toward a domain which is at least 50% identical, at least 60% identical, at least 70% identical, at least 80% identical, at least 90% identical, or at least 95% or 99% identical to a domain of CD47 or SIRPα. The small molecule activators of the present invention may also bind to a particular motif or consensus sequence derived from a particular domain of CD47 or SIRPα, allowing the small molecule activators to specifically bind domains which are shared among members of the CD47 or SIRPα family. In another embodiment, small molecules of the present invention bind protein motifs or consensus sequences which represent the three dimensional structure of the protein. Such motifs or consensus sequences would not represent a contiguous string of amino acids, but a non-linear amino acid arrangement that results from the three-dimensional folding of CD47 or SIRPα (i.e., a structural motif). Such motifs and consensus sequences may be designed according to the any methods known in the art.

### D. Fusion Proteins or Protein Variants

In some embodiments, agents that enhance CD47-SIRPα signaling for use in the methods of the present invention also include variants of CD47 and/or SIRPα protein that can function as agonists and activate the activity of CD47 and/or SIRPα. For example, an agent suitable for use in the methods of the present invention is a full-length CD47 or SIRPα protein, or a fragment of CD47 or SIRPα. In some embodiments, an agent is a wild type CD47 and/or SIRPα protein. In other embodiments, a CD47 and/or SIRPα protein variant or fragment that has an increased activity would also function as an agent for use in the methods of the present invention.

Alternatively, a fusion protein of CD47 and/or SIRPα which functions to increase the expression and/or activities of CD47 and/or SIRPα can also be used in the methods of the present invention. A recombinant fusion protein for CD47 and/or SIRPα for use in the methods of the present invention may be generated from a recombinant vector according to methods know in the art. The recombinant vectors can comprise a nucleic acid encoding a CD47 and/or SIRPα protein in a form suitable for expression of the nucleic acid in a host cell. In some embodiments, a nucleic acid sequence encoding CD47 can be operably linked in an expression vector to a nucleic acid molecule encoding SIRPα, or a functional segment thereof. The resulting fusion protein can then be readily purified from the cells by art recognized methods.

In some embodiments, the recombinant vectors may include one or more regulatory sequences, selected on the basis of the host cells to be used for expression, which is operably linked to the nucleic acid sequence to be expressed (i.e., a recombinant expression vector). Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, CA (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide, or functional fragment thereof, in prokaryotic (e.g., E. coli) or eukaryotic cells (e.g., insect cells using baculovirus expression vectors, yeast cells or mammalian cells). Suitable host cells may include, but not limited to E. coli cells, Bacillus cells, Saccharomyces cells, Pochia cells, NS0 cells, COS cells, Chinese hamster ovary (CHO) cells, myeloma cells, or cells as described herein.

Another aspect of the invention pertains to host cells into which a recombinant vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation.

### E. Screening Assays

Agents that enhance CD47-SIRPα signaling for use in the methods of the present invention can be known or can be identified using the methods described herein. The invention provides methods (also referred to herein as “screening assays”) for identifying the agents, i.e., candidate or test compounds or modulators (e.g., peptides, small molecules or other drugs) which increase the expression and/or activity of CD47 and/or SIRPα and for testing or optimizing the activity of other modulators.

In some embodiments, molecules which bind CD47 and/or its binding ligands, e.g., SIRPα, or have a stimulatory or inhibitory effect on the expression and/or activity of CD47 and/or its binding ligands, e.g., SIRPα, can be identified.

In one embodiment, the ability of a compound to directly modulate the expression, post-translational modification, or activity of CD47 and/or its binding ligands, e.g., SIRPα, is measured as an indicator using a screening assay of the invention.

Examples of agents, modulators, candidate compounds or test compounds include, but are not limited to, nucleic acids (e.g., DNA and RNA), carbohydrates, lipids, proteins, peptides, peptidomimetics, small molecules and other drugs. Agents or modulators can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. Nos. 5,738,996; and 5,807,683, the entire contents of each of the foregoing references are incorporated herein by reference). Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, the entire contents of each of the foregoing references are incorporated herein by reference. Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310). The entire contents of each of the foregoing references are incorporated herein by reference.

The test compound can be contacted with a cell that expresses the CD47 protein or a molecule with which Siglec 15 directly interacts, e.g., SIRPα, For example, the test compound can be contacted with a cell that naturally expresses or has been engineered to express the protein(s) by introducing into the cell an expression vector encoding the protein.

Alternatively, the test compounds can be subjected to a cell-free composition that includes the protein(s) (e.g., a cell extract or a composition that includes e.g., purified natural or recombinant protein).

Compounds that modulate expression and/or activity of CD47 and/or its binding ligands, e.g., SIRPα, can be identified using various “read-outs.” For example, a cell can be transfected with an expression vector, incubated in the presence and in the absence of a test compound, and the effect of the compound on the expression of CD47 and/or its binding ligands, e.g., SIRPα, or on a biological response regulated by CD47 and SIRPα can be determined. The biological activities of CD47 and/or its binding ligands, e.g., SIRPα, include activities determined in vivo, or in vitro, according to standard techniques. Activity can be a direct activity, such as an association between CD47 and SIRPα. Alternatively, the activity is an indirect activity, such as a decrease in cell phagocytosis.

To determine whether a test compound modulates CD47 and/or SIRPα protein expression, in vitro transcriptional assays can be performed. To determine whether a test compound modulates CD47 and/or SIRPα mRNA expression, various methodologies can be performed, such as quantitative or real-time PCR.

A variety of reporter genes are known in the art and are suitable for use in the screening assays of the invention. Examples of suitable reporter genes include those which encode chloramphenicol acetyltransferase, beta-galactosidase, alkaline phosphatase, green fluorescent protein, or luciferase. Standard methods for measuring the activity of these gene products are known in the art.

A variety of cell types are suitable for use as an indicator cell in the screening assay. Preferably a cell line is used which expresses low levels of endogenous CD47 and/or SIRPα and is then engineered to express recombinant protein. Cells for use in the subject assays include eukaryotic cells. For example, in one embodiment, a cell is a fungal cell, such as a yeast cell. In another embodiment, a cell is a plant cell. In yet another embodiment, a cell is a vertebrate cell, e.g., an avian cell or a mammalian cell (e.g., a murine cell, or a human cell).

Recombinant expression vectors that can be used for expression of, e.g., CD47 and/or SIRPα, are known in the art. For example, the cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for or a molecule in a signal transduction pathway involving (e.g., human, murine and yeast) are known in the art and can be used for the design of PCR primers that allow for amplification of a cDNA by standard PCR methods or for the design of a hybridization probe that can be used to screen a cDNA library using standard hybridization methods.

In another embodiment, the test compounds can be subjected to a cell-free composition that includes the protein(s) (e.g., a cell extract or a composition that includes e.g., either purified natural or recombinant protein). CD47 and/or SIRPα expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, compounds that specifically modulate the activity of CD47 and/or SIRPα activity in a signal transduction pathway involving CD47 and/or SIRPα are identified based on their ability to modulate the interaction between CD47 and SIRPα. Suitable assays are known in the art that allow for the detection of protein-protein interactions (e.g., immunoprecipitations, two-hybrid assays and the like). By performing such assays in the presence and absence of test compounds, these assays can be used to identify compounds that enhance the activity of CD47 and/or its binding ligands, e.g., SIRPα.

Compounds identified in the subject screening assays can be used in methods of modulating one or more of the biological responses regulated by CD47 and/or SIRPα. It will be understood that it may be desirable to formulate such compound(s) as pharmaceutical compositions as described herein prior to contacting them with cells.

Once a test compound is identified that directly or indirectly modulates, e.g., CD47 and/or SIRPα expression or activity by one of the variety of methods described hereinbefore, the selected test compound (or “compound of interest”) can then be further evaluated for its effect on cells, for example by contacting the compound of interest with cells either in vivo (e.g., by administering the compound of interest to an organism) or ex vivo (e.g., by isolating cells from an organism and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

In another aspect, the invention pertains to a combination of two or more of the assays described herein. For example, a modulator can be identified using a cell-based or a cell-free assay, and the ability of the modulators to increase the activity of CD47 and/or SIRPα can be confirmed in vivo, e.g., in an animal, such as, for example, an animal model for, e.g., a retinitis pigmentosa mouse model.

Moreover, a modulator of CD47 and/or SIRPα identified as described herein (e.g., a specific antibody, or a small molecule) can be used in an animal model to determine the efficacy, toxicity, or side effects of treatment with such a modulator. Alternatively, a modulator identified as described herein can be used in an animal model to determine the mechanism of action of such a modulator.

Compounds identified by the screening assays of the present invention are considered as candidate therapeutic compounds useful for treating diseases, e.g., degenerative disorders, e.g., degenerative ocular disorders, as described herein. Thus, the invention also includes compounds identified in the screening assays, and methods for their administration and use in the treatment, prevention, or delay of development or progression of diseases described herein.

### IV. Pharmaceutical Compositions of the Invention

In one aspect of the invention, the compositions of the invention will be in the form of a pharmaceutical composition containing a pharmaceutically acceptable carrier. As used herein “pharmaceutically acceptable carrier” refers to any substantially non-toxic carrier conventionally useable for administration of pharmaceuticals in which the isolated polypeptide of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Suitable pharmaceutically acceptable carriers include any and all solvents, dispersion media, coatings, antibacterial and antifungal agents, isotonic and absorption delaying agents, and the like that are physiologically compatible. Pharmaceutically acceptable carriers also include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the gene therapy vector, use thereof in the pharmaceutical compositions of the invention is contemplated. Supplementary active compounds can also be incorporated into the compositions.

Pharmaceutical compositions of the invention may be formulated for delivery to animals for veterinary purposes (e.g. livestock (cattle, pigs, dogs, mice, rats), and other non-human mammalian subjects, as well as to human subjects.

In a particular embodiment, the pharmaceutical compositions of the present invention are in the form of injectable compositions. The compositions can be prepared as an injectable, either as liquid solutions or suspensions. The preparation may also be emulsified. Suitable excipients are, for example, water, saline, dextrose, glycerol, ethanol, phosphate buffered saline or the like and combinations thereof. In addition, if desired, the preparation may contain minor amounts of auxiliary substances such as wetting or emulsifying agents, pH-buffering agents, adjuvants, surfactant or immunopotentiators.

In a particular embodiment, the compositions of the invention are incorporated in a composition suitable for intraocular administration. For example, the compositions may be designed for intravitreal, subretinal, subconjuctival, sub-tenon, periocular, retrobulbar, suprachoroidal, and/or intrascleral administration, for example, by injection, to effectively treat the retinal disorder. Additionally, a sutured or refillable dome can be placed over the administration site to prevent or to reduce “wash out”, leaching and/or diffusion of the active agent in a non-preferred direction.

Relatively high viscosity compositions, as described herein, may be used to provide effective, and preferably substantially long-lasting delivery of the nucleic acid molecules and/or vectors, for example, by injection to the posterior segment of the eye. A viscosity inducing agent can serve to maintain the nucleic acid molecules and/or vectors in a desirable suspension form, thereby preventing deposition of the composition in the bottom surface of the eye. Such compositions can be prepared as described in U.S. Pat. No. 5,292,724, the entire contents of which are hereby incorporated herein by reference.

Sterile injectable solutions can be prepared by incorporating the compositions of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation include vacuum drying and freeze-drying which yields a powder of the active ingredient plus any additional desired ingredient from a previously sterile-filtered solution thereof.

Toxicity and therapeutic efficacy of nucleic acid molecules described herein can be determined by standard pharmaceutical procedures in cell cultures or experimental animals, e.g., for determining the ED₅₀ (the dose therapeutically effective in 50% of the population). Data obtained from cell culture assays and/or animal studies can be used in formulating a range of dosage for use in humans. The dosage typically will lie within a range of concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays.

This invention is further illustrated by the following examples which should not be construed as limiting. The contents of all references, patents and published patent applications cited throughout this application, as well as the Figures, are hereby incorporated by reference.

### EXAMPLES

### Experimental Model and Subject Details

### Mice.

All animal experiments were conducted under protocols approved by the Institutional Animal Care and Use Committee (IACUC) of Harvard Medical School. CD-1 (#022) and FVB (#207) mice were purchased from Charles River Laboratories. CX3CR1^{GFP} (#005582), rd10 (#004297), C3H (#000659), sighted C3H (#003648), R26-CreERT2 (#008463), and TSP1^{-/-} (#006141) mice were purchased from The Jackson Laboratory. Rho^{-/-} mice were a gift from Janis Lem (Tufts University) (Lem et al., 1999). SIRPα^{-/-} mice were a gift from Beth Stevens (Harvard Medical School) (Lehrman et al., 2018). CX3CR1^{GFP}, R26-CreERT2, TSP1^{-/-}, and SIRPα^{-/-} lines were crossed with FVB mice for at least four generations to obtain the following strains: sighted CX3CR1^{GFP/+}, rd1;CX3CR1^{GFP/+}, rd1;CreERT2/+, rd1;TSP1^{+/-}, rd1;TSP1^{-/-}, rd1;SIRPα^{+/-}, and rd1;SIRPα^{-/-}. Genotyping was performed by Transnetyx using real-time PCR.

Mice were maintained at Harvard Medical School on a 12-hour alternating light and dark cycle. Animals were housed in standard ventilated racks at a density of up to five per cage. Both males and females were used in all experiments and were randomly assigned to experimental groups.

### Histology.

Retinal flat-mounts from rd1 (FVB), rd10, and Rho^{-/-} mice were prepared as previously described (Wang et al., 2019, 2020a). Following dissection of enucleated eyes in phosphate-buffered saline (PBS), retinas were fixed in 4% paraformaldehyde for 30 minutes at room temperature, washed twice with PBS, and relaxed with four radial incisions. For cone arrestin immunostaining, retinas were additionally blocked with 5% donkey serum and 0.3% Triton X-100 in PBS for one hour at room temperature, incubated with anti-cone arrestin (1:3000) in blocking solution overnight at 4° C., and labeled with donkey anti-rabbit secondary (1:1000) in PBS for two hours at room temperature. Retinas were mounted on microscope slides using Fluoromount-G (SouthernBiotech) with the ganglion cell layer facing up.

For retinal cross-sections, enucleated eyes were dissected to remove the cornea, iris, lens, and ciliary body. The remaining eye cups were cryoprotected in a sucrose gradient, frozen in a 1:1 mixture of optimal cutting temperature compound (Tissue-Tek) and 30% sucrose in PBS, and sectioned on a Leica CM3050S cryostat (Leica Microsystems) at a thickness of 20 μm. Tissues were blocked with 5% donkey serum and 0.1% Triton X-100 in PBS for one hour at room temperature and incubated with anti-CD47 (1:1000), anti-TSP1 (1:500), or anti-SIRPα (1:500) in blocking solution overnight at 4° C. Sections were subsequently labeled with the appropriate secondary antibody (1:1000) in PBS for two hours at room temperature followed by 0.5 μg/mL of 4′,6-diamidino-2-phenylindole (DAPI) (ThermoFisher Scientific) in PBS for five minutes at room temperature prior to mounting.

### Ex Vivo Phagocytosis Assay.

Freshly dissected retinas were incubated with gentle agitation for one hour at 37° C. in 1 mg/mL of pHrodo Red-conjugated zymosan bioparticles resuspended in retinal culture media (1:1 mixture of DMEM and F-12 supplemented with L-glutamine, B27, N2, and penicillin-streptomycin). Retinas were subsequently washed with PBS and dissociated using cysteine-activated papain as previously described (Wang et al., 2019). After two additional washes, samples were passed through a 40 m filter and stained with 0.5 μg/mL of DAPI in FACS buffer (2 mM EDTA and 2% fetal bovine serum in PBS) to exclude non-viable cells. Data were collected on a Cytek DxP11 cytometer and analyzed using FlowJo 10. CX3CR1-positive microglia were defined as phagocytic if positive for pHrodo Red.

### AAV Vector Cloning and Production.

The AAV-human red opsin-GFP-WPRE-bGH (AAV8-RedO-GFP) plasmid was a gift from Botond Roska (Institute for Molecular and Clinical Ophthalmology Basel) (Busskamp et al., 2010). The AAV8-RedO-CD47 plasmid was cloned by replacing the GFP coding sequence in AAV8-RedO-GFP with that of the most abundant isoform of mouse CD47 (NM_010581.3) (Brown and Frazier, 2001). The AAV8-RedO-FLEX-mCherry plasmid was cloned by replacing the GFP coding sequence in AAV8-RedO-GFP with that of mCherry inverted and flanked by lox2272 and loxP sites. The AAV8-RedO-FLEX-CD47 plasmid was cloned by replacing the inverted mCherry sequence in AAV8-RedO-FLEX-mCherry with that of inverted CD47.

AAV vectors were generated as previously described by transfecting 293T cells with a mixture the vector plasmid, adenovirus helper plasmid, and rep2/cap8 packaging plasmid (Grieger et al., 2006; Xiong et al., 2019). Seventy-two hours post-transfection, viral particles were harvested from the supernatant, PEGylated overnight, precipitated by centrifugation, treated with Benzonase nuclease (Sigma-Aldrich), and purified through an iodixanol gradient before collection in 100-200 μL of PBS. Vectors were semi-quantitatively titered by SYPRO Ruby (Molecular Probes) for viral capsid proteins (VP1, VP2, and VP3) relative to a reference vector titered by real-time PCR

### AAV Vector Delivery.

Subretinal injections of AAV vectors were performed on neonatal mice (PO-1) as previously described (Wang et al., 2020b). Following anesthetization of the animal on ice, the palpebral fissure was gently opened with a 30-gauge needle and the eye exposed. Using a glass needle controlled by a FemtoJet microinjector (Eppendorf), ˜0.25 μL of vectors were then delivered into the subretinal space. Both left and right eyes were used for injections. For AAV8-RedO-GFP, ˜5×10⁸ vector genomes (vg) per eye were administered. All other vectors were dosed at −1×10⁹ vg per eye.

### Image Acquisition and Analysis.

Retinal flat-mounts were imaged using a Nikon Ti inverted widefield microscope (10× or 20× air objective). Retinal cross-sections were imaged using a Zeiss LSM710 scanning confocal microscope (20× air objective or 40× oil objective). All image analysis was performed using ImageJ. To quantify GFP-positive or cone arrestin-positive cones, custom ImageJ modules were used as previously described (Wang et al., 2019, 2020a). For each flat-mount, the locations of the optic nerve head and four retinal leaflets were first manually defined. The number of GFP-positive or cone arrestin-positive objects within the region corresponding to the central retina was then automatically counted and used to represent the number of cones in that sample.

### Light-Dark Test.

Light avoidance in rd1 (C3H) and sighted C3H mice following no treatment or treatment in both eyes was assessed as previously described (Wang et al., 2020a). A plastic chamber (Med Associates) measuring 28 cm (length) by 28 cm (width) by 21 cm (height) was divided into two equally sized compartments: one dark and one brightly illuminated (˜900 lux). The two compartments were connected by a small opening and differed in temperature by less than 1° C. At the start of each trial, a mouse was placed in the illuminated compartment and its activity recorded for ten minutes. If after one minute, the animal had not yet entered the dark compartment, it was gently guided there, removed from the chamber, and the trial restarted. Mice were tracked using infrared sensors and location data analyzed with Activity Monitor (Med Associates). Percent time spent in dark was calculated based on the final nine minutes of each trial.

### Optomotor Assay.

Visual acuity was measured by an observer (Y.X.) blinded to the treatment groups using the OptoMotry System (CerebralMechanics) as previously described (Wang et al., 2019). Animals were placed in a chamber with bright background luminance to saturate rods and presented with moving gratings of varying spatial frequencies. Left and right eyes were assessed using clockwise and counterclockwise gratings, respectively, as the optomotor response is evoked by temporal-to-nasal motion in mice (Douglas et al., 2005). For each eye, the highest spatial frequency at which the mouse tracked the grating was determined to be the visual acuity.

### Tamoxifen Injections.

Tamoxifen was dissolved in corn oil (Sigma-Aldrich) and dosed at 2 mg daily from P19-21 via intraperitoneal injections.

### Microglia Depletion.

Microglia were depleted using PLX5622, an orally available CSF1R inhibitor. PLX5622 was formulated into AIN-76A rodent chow (Research Diets) at 1200 mg/kg and provided ad libitum during periods of depletion. For quantification of retinal microglia, freshly dissected retinas were dissociated using cysteine-activated papain as previously described (Wang et al., 2019). Cells were subsequently blocked with anti-CD16/32 (1:100) for five minutes on ice followed by incubation with PE-Cy5-conjugated anti-CD11b (1:200), APC-Cy7-conjugated anti-Ly6C (1:200), and APC-Cy7-conjugated anti-Ly6G (1:200) for 20 minutes on ice. After washes, samples were passed through a 40 m filter and stained with 0.5 μg/mL of DAPI in FACS buffer. Data were collected on a BD FACSAria II and analyzed using FlowJo 10.

### Quantification and Statistical Analysis

Statistical analyses were performed using GraphPad Prism software. Experimental groups were compared using two-tailed Student's t-tests, with the addition of a Bonferroni correction if three or more comparisons were performed. A P value of less than 0.05 was considered statistically significant. Information on group data and replicates is reported in each figure legend.

### Example 1. Microglia Show Increased Phagocytic Activity During Secondary Cone Degeneration

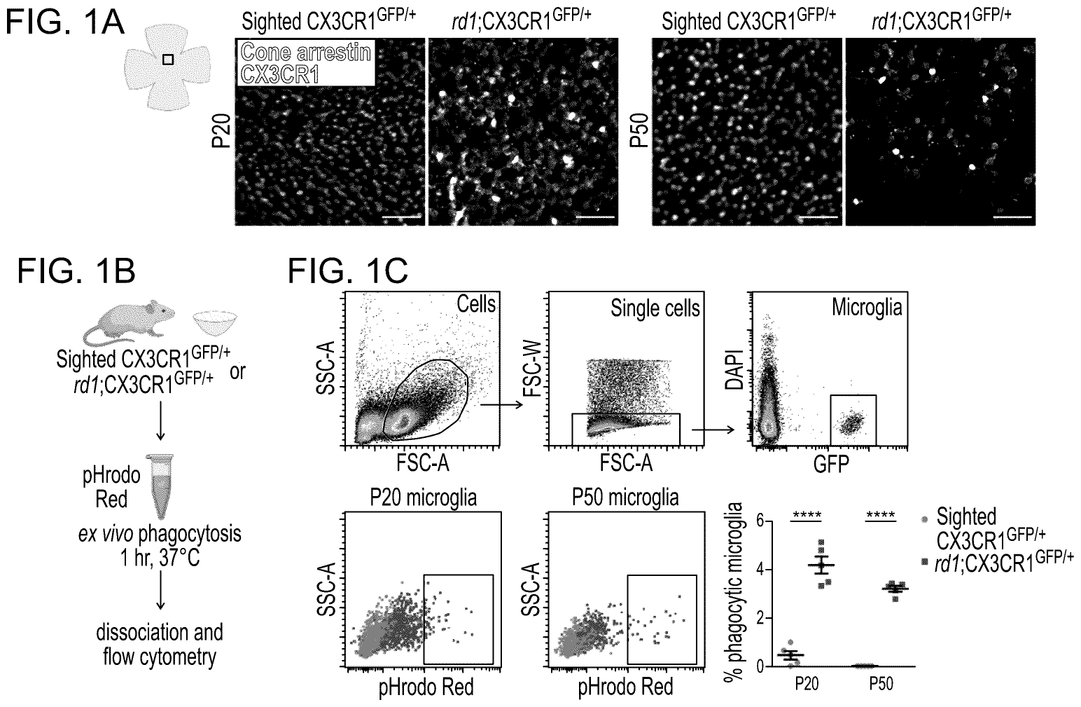

rd1;CX3CR1^{GFP/+} and sighted CX3CR1^{GFP/+} (rd1 heterozygous) mice were generated by crossing the widely used rd1 model of RP with CX3CR1^{GFP} animals, in which microglia express GFP (Chang et al., 2002; Jung et al., 2000; Wang et al., 2019). To assess if microglia during the later stages of RP might phagocytose cones, retinas from these mice were immunostained for cone arrestin, a marker of all cones. In sighted CX3CR1^{GFP/+} retinas, microglia lacked any appreciable contact with cone arrestin-positive cells (FIG. 1A), consistent with normal exclusion of microglia from the photoreceptor layer (O'Koren et al., 2019). In contrast, microglia in rd1;CX3CR1^{GFP/+} retinas often directly interfaced with degenerating cones, although in no case was overt engulfment of cones observed. To more sensitively measure microglial phagocytic activity, retinas from rd1;CX3CR1^{GFP/+} and sighted CX3CR1^{GFP/+} animals were explanted and incubated them with yeast particles conjugated to pHrodo Red, a pH-dependent dye that fluoresces upon lysosomal acidification (FIG. 1B) (Miksa et al., 2009). Microglia from these retinas were then analyzed by flow cytometry at postnatal day 20 (P20), the approximate age at which cone death in rd1 mice begins (Punzo et al., 2009), as well as P50, after substantial cone loss has occurred. At both time points, microglia from rd1;CX3CR1^{GFP/+} retinas internalized significantly more yeast than those from sighted controls (FIG. 1C), suggesting persistent elevation of microglial uptake during the later stages of RP. Microglia thus exhibit increased phagocytic activity during secondary cone degeneration, a factor which may worsen cone demise.

### Example 2. Expression of the CD47 “Don't Eat Me” Signal Promotes Survival of Degenerating Cones

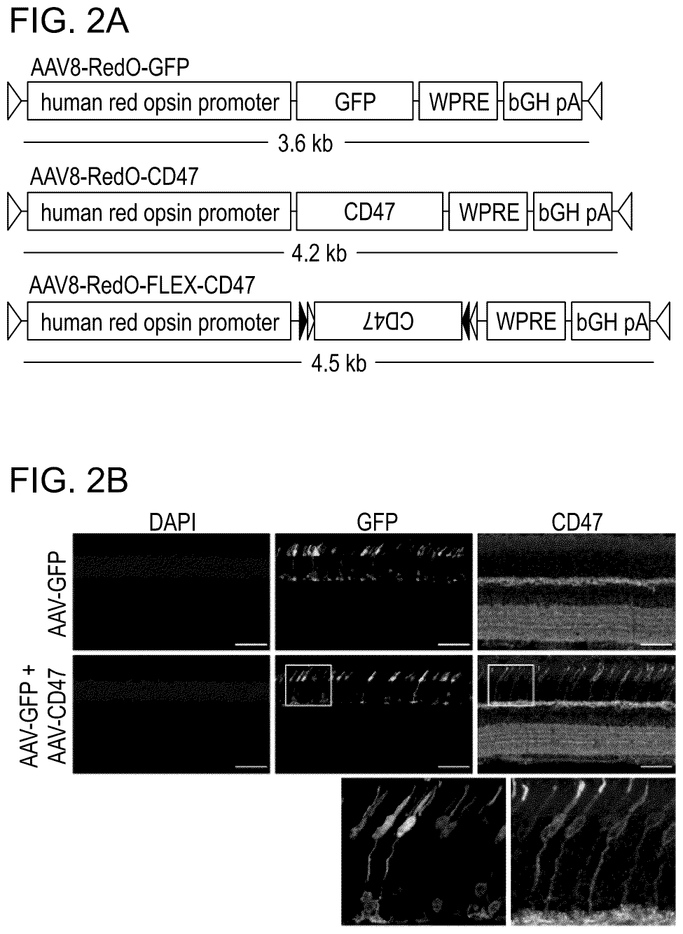

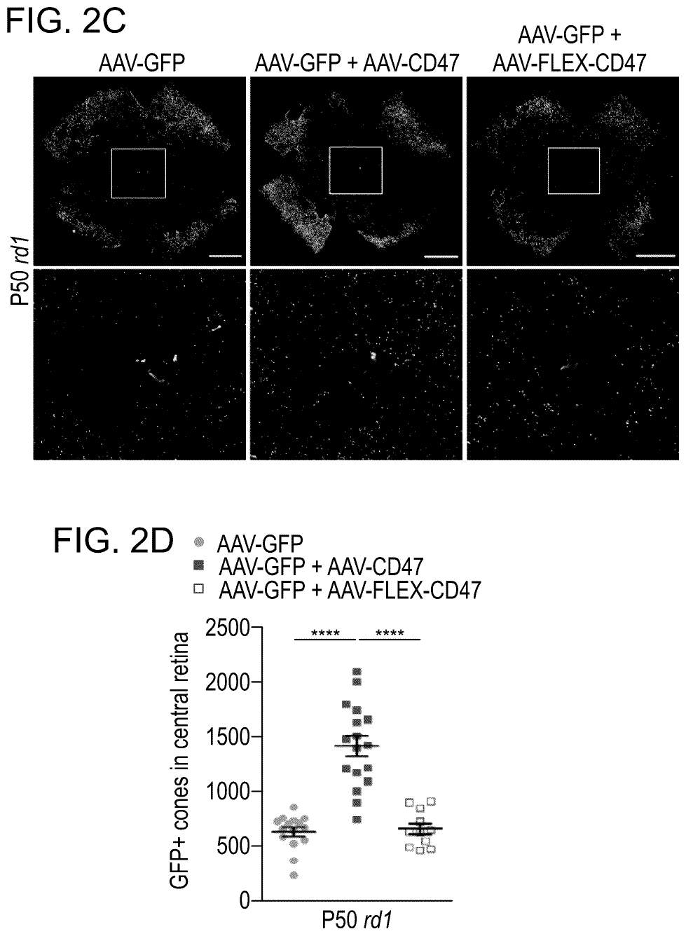

Among the key regulators of phagocytosis are “don't eat me” signals such as CD47, which when present on cells, impede their engulfment by macrophages (Park and Kim, 2017). To test if inhibiting phagocytosis during RP might benefit cones, an AAV vector (AAV8-RedO-CD47) was created using the human red opsin promoter to express CD47 on cones (FIG. 2A). In wild-type mice injected subretinally with a GFP control vector (AAV8-RedO-GFP), which clearly labeled transduced cones, endogenous CD47 could be seen in retinal plexiform layers as previously described, but not in photoreceptors (FIG. 2B) (Mi et al., 2000). Following co-administration of AAV8-RedO-GFP plus AAV8-RedO-CD47, CD47 immunostaining could additionally be detected in cones. Notably, use of AAV8-RedO-CD47 in wild-type mice produced no obvious changes in cone or retinal morphology when evaluated one month later.

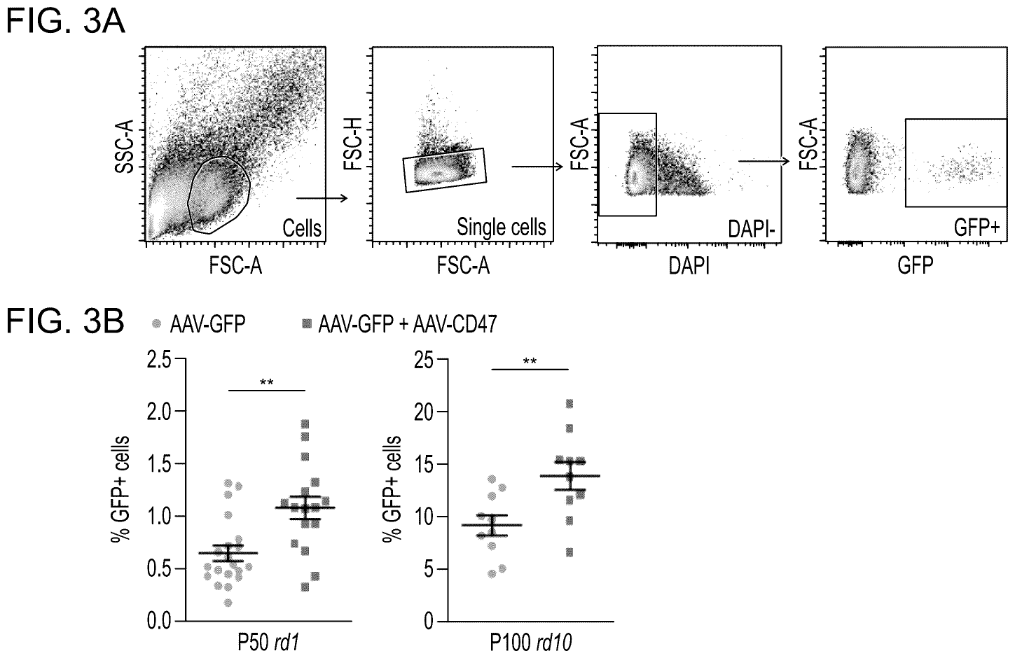

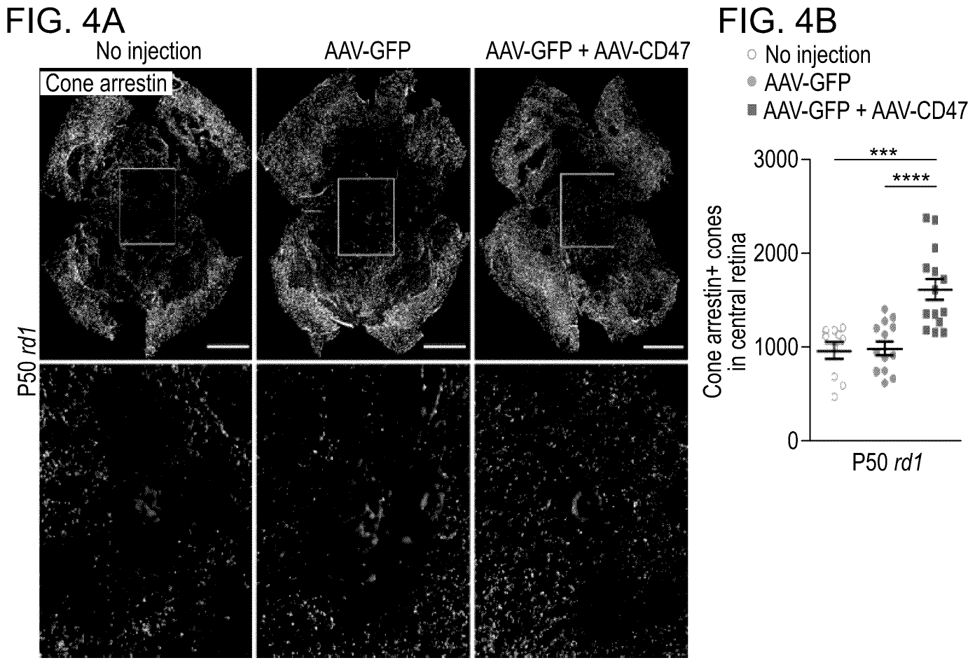

In mouse models of RP, cone degeneration proceeds from the optic nerve head outward with relative sparing of the peripheral retina. To measure the effect of CD47 on cone survival, GFP-positive cones were thus quantified in the central retinas of rd1 mice. Compared to AAV8-RedO-GFP alone, co-treatment with AAV8-RedO-CD47 approximately doubled the number of cones in the central retina at P50 (FIGS. 2C and 2D). In contrast, co-treatment with AAV8-RedO-FLEX-CD47, a control vector with the CD47 sequence inverted, did not significantly change the number of remaining cones. To assess cone preservation with CD47 beyond the central retina, whole rd1 retinas were next analyzed for GFP-positive cones using flow cytometry (FIG. 3A). Consistent with the histological findings, this method showed greater cone counts at P50 with AAV8-RedO-GFP plus AAV8-RedO-CD47 than AAV8-RedO-GFP only (FIG. 3B). To also gauge cone survival versus untreated eyes, rd1 retinas at P50 were immunostained for cone arrestin. Relative to central retinas without treatment or treated with AAV8-RedO-GFP alone, addition of AAV8-RedO-CD47 again resulted in more cones as defined by this marker (FIGS. 4A and 4B). Together, these data demonstrated that CD47 expression could promote survival of cones in rd1 mice.

### Example 3. CD47 Promotes Cone Survival and Retention of Vision in Multiple Genetic Models

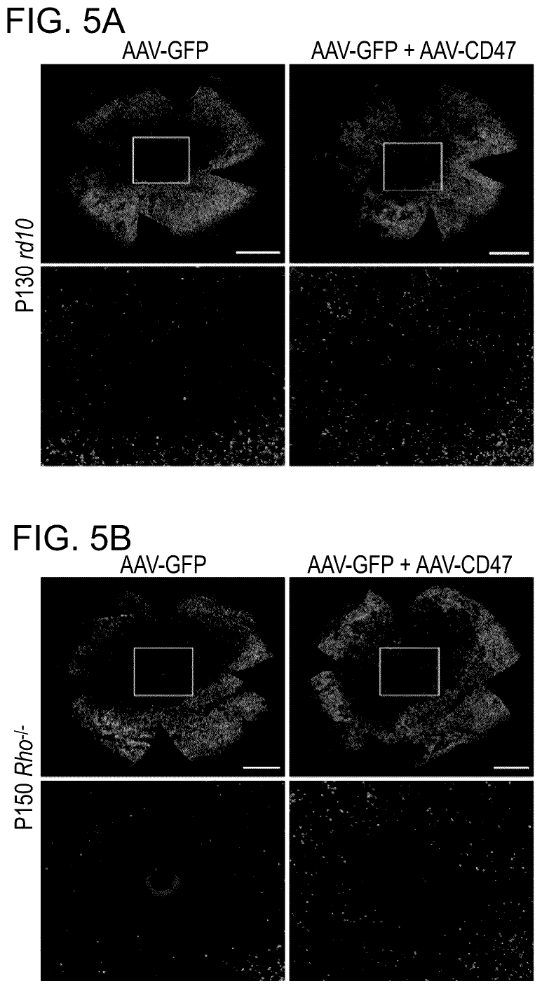

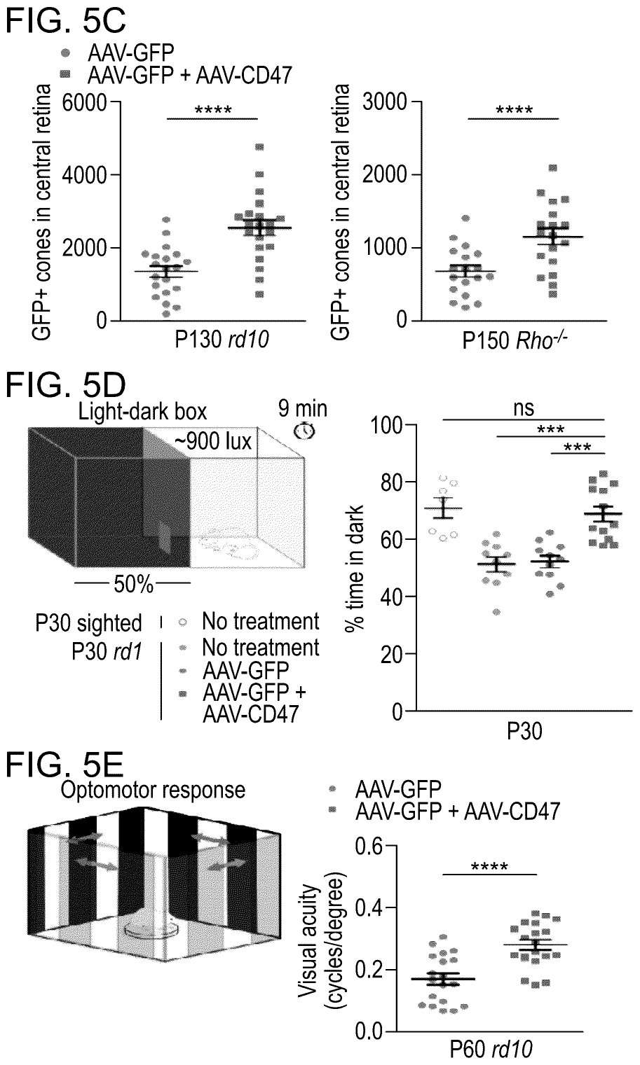

To determine if CD47 might similarly slow retinal degeneration in other models of RP, AAV8-RedO-CD47 was tested in rd10 mice, which carry a missense mutation in Pde6b, and in Rho^{-/-} mice, which lack rhodopsin. In both rd10 retinas at P100 and P130 and Rho^{-/-} retinas at P150, AAV8-RedO-CD47 again improved the number of cones (FIGS. 5A-5C and FIG. 3C), suggesting that CD47 may generically combat cone degeneration.

To investigate the therapeutic relevance of AAV8-RedO-CD47, treated animals were then subjected to two visually dependent behavioral assays. First, a light-dark discrimination test was performed by leveraging the natural preference of sighted mice for dark rather than well-illuminated spaces. Keeping with this, wild-type animals spent ˜70% of their time in the dark half of a 50:50 light-dark environment, while rd1 mice without treatment or receiving only AAV8-RedO-GFP divided their time evenly between the two chambers (FIG. 5D). Compared to these latter two groups, rd1 animals receiving AAV8-RedO-GFP plus AAV8-RedO-CD47 spent significantly more time in the dark, consistent with better preservation of visual function.

As a second measure of vision, mice were evaluated using an optomotor assay in which moving stripes were presented to elicit the visually dependent optomotor response. By varying the spatial frequency of stripes to make them easier or more difficult to see, the visual acuity in each eye could be estimated (Douglas et al., 2005). In rd10 animals tested at P60, visual acuity was significantly higher in eyes treated with AAV8-RedO-GFP plus AAV8-RedO-CD47 than with AAV8-RedO-GFP alone (FIG. 5E). CD47 expression thus not only promotes cone survival in different mouse models of RP, but also protects from vision loss, supporting its potential use as a mutation-agnostic therapy for this condition.

### Example 4. Delayed Expression of CD47 Ameliorates Cone Death

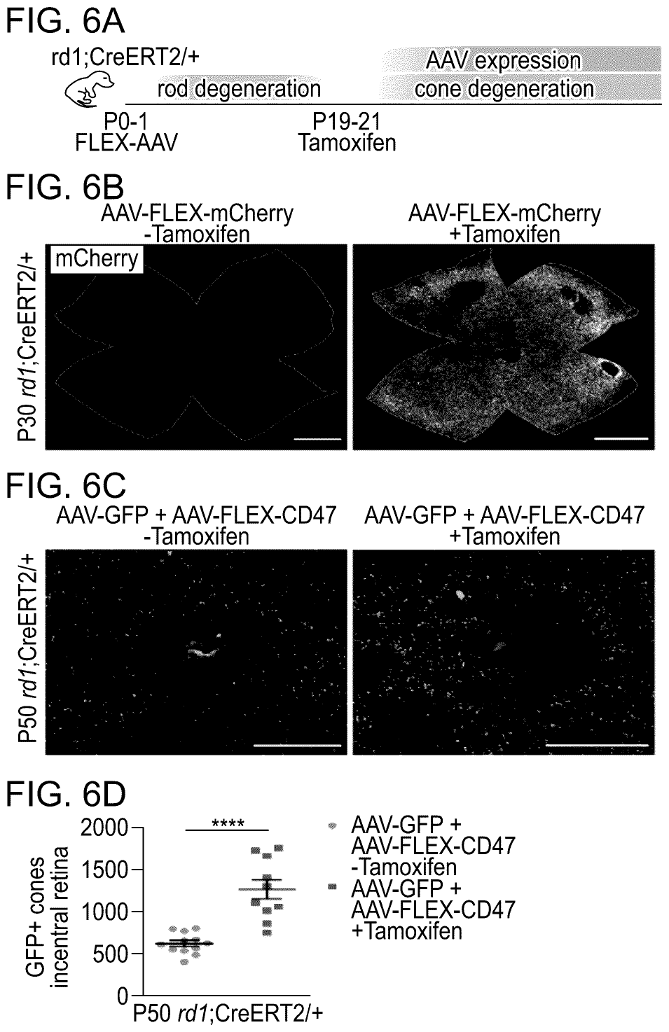

Clinically, patients with RP are often diagnosed following the development of night blindness (Hartong et al., 2006). In order to test whether CD47 could still protect cones even after the majority of rods have died, R26-CreERT2 mice, which undergo inducible Cre activation in the presence of tamoxifen, were breed with the rd1 strain to obtain rd1;CreERT2/+ animals. When combined with a flip-excision (FLEX) vector (Atasoy et al., 2008), this approach allowed for delayed AAV expression while avoiding the technical challenges of subretinal injections in older mice. As a proof-of-concept, PO-1 rd1;CreERT2/+ animals were treated with AAV8-RedO-FLEX-mCherry, a vector designed to express mCherry only after exposure to tamoxifen (FIG. 6A). In the absence of tamoxifen, AAV8-RedO-FLEX-mCherry produced no detectable fluorescence after 30 days (FIG. 6B). However, the same vector with tamoxifen from P19-21 led to robust mCherry expression throughout the retina. Using this strategy and an analogous AAV8-RedO-FLEX-CD47 vector, the effect of delayed CD47 expression on cone survival was examined. In rd1;CreERT2/+ animals treated with AAV8-RedO-GFP plus AAV8-RedO-FLEX-CD47 but without tamoxifen, the number of cones at P50 was comparable to that seen in rd1 mice (FIGS. 2D, 6C, and 6D). In contrast, in animals additionally receiving tamoxifen from P19-21, an age by which most rods have died (Punzo et al., 2009), significantly more GFP-positive cones were observed. Collectively, these findings suggest that CD47 may alleviate cone degeneration even if administered later in the disease progression.

### Example 5. CD47 Requires SIRPα but not Microglia or TSP1 to Preserve Cones

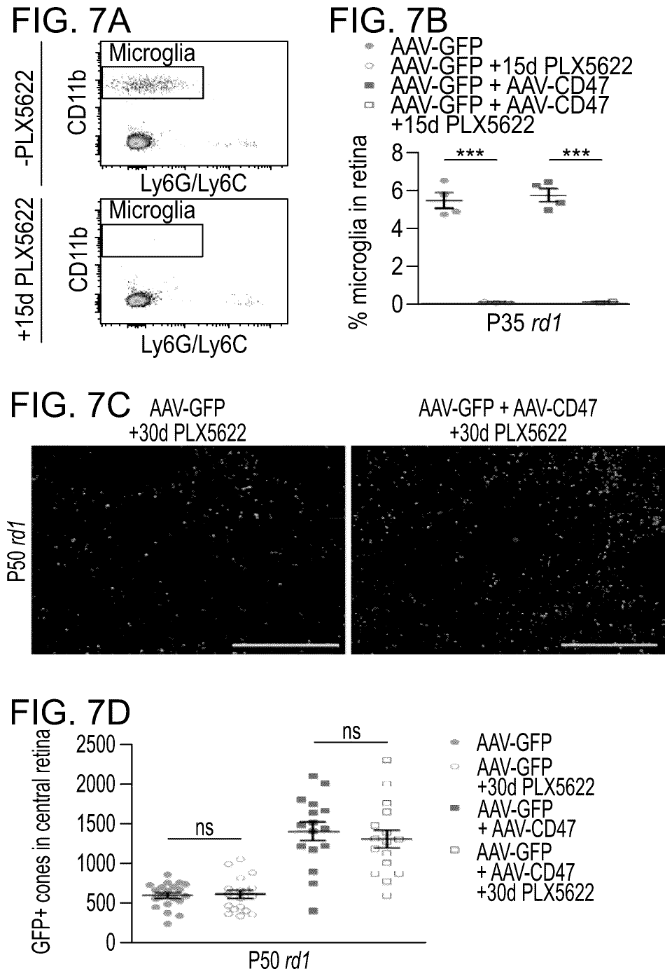

In the brain, the “don't eat me” function of CD47 is thought to be largely mediated by microglia (Hutter et al., 2019; Lehrman et al., 2018). In order to determine whether AAV8-RedO-CD47 might protect cones by inhibiting excess microglial phagocytosis, thereby preventing cones from being pathologically engulfed, microglia were pharmacologically depleted during treatment with AAV8-RedO-CD47 using PLX5622, a small molecule blocking a receptor on microglia essential for their survival (Elmore et al., 2014). In the retina, PLX5622 depleted ˜99% of microglia within 15 days (FIGS. 7A and 7B), indicating virtually complete elimination of this cell type. Consistent with the previous observations (Wang et al., 2020a), PLX5622 administration in rd1 mice from P20-49 had no significant effect on cone counts in eyes receiving only AAV8-RedO-GFP (FIGS. 7C and 7D). Surprisingly, PLX5622 failed to perturb cone preservation with AAV8-RedO-CD47, implying that microglia were dispensable for its mechanism of action.

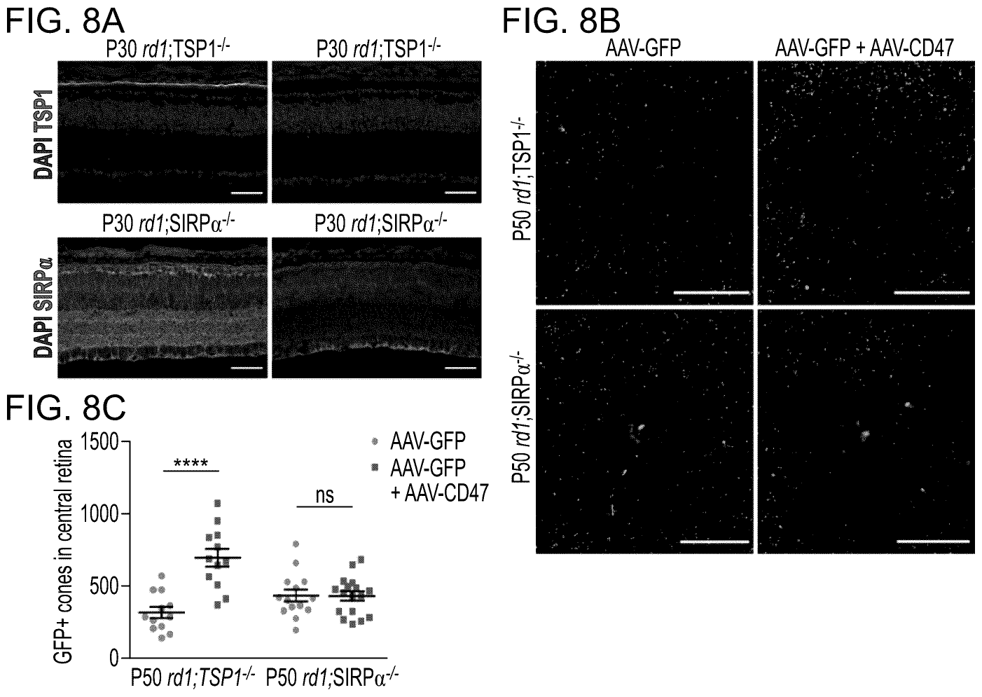

Consequently, two of the major pathways by which CD47 is known to mediate intercellular signaling were examined. Through its extracellular domain, CD47 can interact with SIRPα, a transmembrane protein expressed on a broad range of cell types including microglia, macrophages, dendritic cells, and neurons (Matlung et al., 2017). In addition, CD47 can be activated by thrombospondin-1 (TSP1), a secreted protein whose binding to CD47 has been shown to help resolve subretinal inflammation (Calippe et al., 2017). To determine if either TSP1-CD47 or CD47-SIRPα signaling were necessary for CD47 to save cones, rd1;TSP1^{-/-} and rd1;SIRPα^{-/-} mice, which exhibited loss of retinal TSP1 and SIRPα, respectively, were generated (FIG. 8A). In rd1;TSP1^{-/-} animals, treatment with AAV8-RedO-GFP plus AAV8-RedO-CD47 doubled the number of remaining cones relative to AAV8-RedO-GFP alone (FIGS. 8B and 8C). In contrast, in rd1;SIRPα^{-/-} mice, no difference in cone survival was observed with or without AAV8-RedO-CD47. These results demonstrated that the therapeutic effect of AAV8-RedO-CD47 requires SIRPα but not TSP1. Protection of cones with CD47 therefore likely occurs via increased CD47-SIRPα signaling involving one or more non-microglial cell types.

### DISCUSSION

Despite a growing number of gene therapy programs targeting IRDs, the vast majority of patients still lack effective treatment. Correcting individual genes or mutations may improve this situation, but only incrementally, highlighting the need for an intervention that can benefit all comers. In this application, the inventors developed AAV8-RedO-CD47, a novel gene therapy vector that protected cones and vision in multiple models of RP, supporting its potential use across different mutations. Importantly, delayed expression of CD47 with AAV8-RedO-FLEX-CD47 could also preserve cones, suggesting that this therapy would still be beneficial if administered later in disease, after most rods have died. AAV8-RedO-CD47 thus offers a possible treatment option for many patients with RP, including those who are diagnosed at older ages or with genetics that preclude straightforward gene replacement. The vector may further help combat cone death in other IRDs and degenerative retinal disorders such as age-related macular degeneration (AMD), which affects an estimated 200 million people worldwide (Wong et al., 2014).

A key lingering question is how exactly CD47 makes cones more resistant to degeneration. While the inventors initially hypothesized that better cone survival would be secondary to reduced engulfment by microglia, this explanation was incompatible with the PLX5622 experiments, in which near complete elimination of retinal microglia failed to perturb CD47-mediated protection of cones. Additionally, although cones were exposed to TSP1 secreted from the adjacent retinal pigment epithelium (RPE), AAV8-RedO-CD47 still helped preserve cones in the absence of TSP1-CD47 signaling. Instead, cone rescue with AAV8-RedO-CD47 required SIRPα, indicating that CD47 on cones likely interacts with SIRPα on non-microglial cells to alleviate degeneration. Outside of microglia, SIRPα in the eye is normally expressed in the synapse-rich plexiform layers of the retina (Mi et al., 2000). However, the protein is also present on multiple immune cell populations, including monocytes, macrophages, neutrophils, and dendritic cells, and has more recently been described on natural killer (NK) and a subset of cytotoxic T cells (Deuse et al., 2021; Matlung et al., 2017; Myers et al., 2019). Binding of CD47 to SIRPα on immune cell populations consistently acts as a negative checkpoint, whether by inhibiting phagocytosis, preventing cell killing, or impeding recruitment of adaptive immunity (Matlung et al., 2017). Increased CD47-SIRPα signaling with AAV8-RedO-CD47 may save cones by suppressing dysregulated immune cells that would otherwise contribute to cone demise.

Therapeutic interest in the CD47-SIRPα axis has grown tremendously in recent years following the discovery that many cancers co-opt CD47 expression to evade anti-tumor immunity (Chao et al., 2012). Blocking CD47 and SIRPα have since become promising avenues to induce killing of tumor cells (Weiskopf et al., 2013; Willingham et al., 2012), with several modalities now being trialed in patients (Advani et al., 2018; Sikic et al., 2019). Rather than disrupt, the inventors augmented CD47-SIRPα signaling in the eye by using an AAV vector to overexpress CD47. This approach was able to slow progression in multiple models of a blinding disease and may likewise be applicable to other neurodegenerative conditions. For example, CD47 levels have been found to be low in active lesions during multiple sclerosis (Han et al., 2012), hinting that increasing CD47 in these regions might ameliorate neuroinflammation. It would similarly be interesting to see if CD47 overexpression could aid other cell types that undergo degeneration such as retinal ganglion cells in glaucoma or motor neurons in amyotrophic lateral sclerosis (ALS).

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for treating or preventing a degenerative ocular disorder in a subject, comprising administering to the subject a therapeutically effective amount of an agent that enhances CD47-SIRPα signaling, thereby treating or preventing the degenerative ocular disorder in the subject.

2. The method of claim 1, wherein loss of functional vision in the subject is prevented.

3. A method for prolonging the viability of a photoreceptor cell compromised by a degenerative ocular disorder, comprising contacting the cell with an agent that enhances CD47-SIRPα signaling, thereby prolonging the viability of the photoreceptor cell compromised by the degenerative ocular disorder.

4. The method of claim 3, wherein the contacting occurs in vitro; or wherein the cell is within a subject.

5. (canceled)

6. The method of claim 1, wherein the subject is a human subject.

7. The method of claim 1,
(a) wherein the degenerative ocular disorder is associated with decreased viability of cone cells and/or decreased viability of rod cells;
(b) wherein the degenerative ocular disorder is selected from the group consisting of retinitis pigmentosa, age related macular degeneration, cone rod dystrophy, rod cone dystrophy and glaucoma;
(c) wherein the degenerative ocular disorder is a genetic disorder;
(d) wherein the degenerative ocular disorder is not associated with blood vessel leakage and/or growth; and/or
(e) wherein the degenerative ocular disorder is retinitis pigmentosa.

8-11. (canceled)

12. The method of claim 1,
(a) wherein the agent prevents degeneration of a cone photoreceptor cell;
(b) wherein the agent increases the expression and/or activity of CD47 and/or SIRPα;
(c) wherein the agent is selected from the group consisting of a viral vector construct expressing CD47, an adeno-associated virus (AAV) expression cassette comprising a nucleic acid molecule encoding CD47; a small molecule activator of CD47; an anti-CD47 agonistic antibody or antigen-binding fragment thereof; a CD47 fusion protein; a CD47 protein or fragment thereof; an AAV expression cassette comprising a nucleic acid molecule encoding SIRPα; a small molecule activator of SIRPα; an anti-SIRPα agonistic antibody or antigen-binding fragment thereof; a SIRPα fusion protein; and a SIRPα protein or fragment thereof; and/or
(d) wherein the agent is an AAV expression cassette comprising a nucleic acid molecule encoding CD47.

13. (canceled)

14. (canceled)

15. The method of claim 12, wherein the agent is an AAV expression cassette comprising a nucleic acid molecule encoding CD47;
(a) wherein the nucleic acid molecule encoding CD47 comprises a nucleotide sequence of any one of SEQ ID NOs:1-8; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of any one of SEQ ID NOs:1-8;
(b) wherein the nucleic acid molecule encoding CD47 comprises a nucleotide sequence of SEQ ID NO:7; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:7;
(c) wherein the AAV expression cassette comprises a promoter selected from the group consisting of a human red opsin (hRedO) promoter, a human Best1 promoter, a CMV promoter, a CAG promoter and a human Rhodopsin promoter;
(d) wherein the AAV expression cassette further comprises a Kozak sequence;
(e) wherein the AAV expression cassette further comprises a Woodchuck hepatitis virus posttranscriptional regulatory element (WPRE);
(f) wherein the AAV expression cassette further comprises a polyadenylation signal; and/or
(g) wherein the expression cassette is present in an AAV vector selected from the group consisting of AAV2, AAV 8, AAV2/5, and AAV 2/8.

16-19. (canceled)

20. The method of claim 15,
(a) wherein the human red opsin (hRedO) promoter comprises the nucleotide sequence of SEQ ID NO: 9, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:9;
(b) wherein the human Best 1 promoter comprises the nucleotide sequence of SEQ ID NO: 10, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 10;
(c) wherein the CMV promoter comprises the nucleotide sequence of SEQ ID NO: 11, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 11;
(d) wherein the CAG promoter comprises the nucleotide sequence of SEQ ID NO: 12, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 12;
(e) wherein the human Rhodopsin promoter comprises the nucleotide sequence of SEQ ID NO: 13, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 13;
(f) wherein the Kozak sequence comprises the nucleotide sequence of GCCGCCACC, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of GCCGCCACC;
(g) wherein the WPRE comprises the nucleotide sequence of SEQ ID NO: 15, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 15; and/or
(h) wherein the polyadenylation signal comprises the nucleotide sequence of SEQ ID NO: 16, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 16.

21-34. (canceled)

35. A composition, comprising an adeno-associated virus (AAV) expression cassette, the expression cassette comprising a promoter and a nucleic acid molecule encoding CD47.

36. The composition of claim 35,
(a) wherein the nucleic acid molecule encoding CD47 comprises a nucleotide sequence of any one of SEQ ID NOs:1-8; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of any one of SEQ ID NOs:1-8, or
(b) wherein the nucleic acid molecule encoding CD47 comprises a nucleotide sequence of SEQ ID NO: 7; or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 7.

37. (canceled)

38. The composition of claim 35, wherein the promoter is selected from a group consisting of a human red opsin (hRedO) promoter, a human Best1 promoter, a CMV promoter, a CAG promoter and a human Rhodopsin promoter.

39. The composition of claim 38,
(a) wherein the human red opsin (hRedO) promoter comprises the nucleotide sequence of SEQ ID NO: 9, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO:9;
(b) wherein the human Best 1 promoter comprises the nucleotide sequence of SEQ ID NO: 10, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 10;
(c) wherein the CMV promoter comprises the nucleotide sequence of SEQ ID NO: 11, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 11;
(d) wherein the CAG promoter comprises the nucleotide sequence of SEQ ID NO: 12, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 12; and/or
(e) wherein the human Rhodopsin promoter comprises the nucleotide sequence of SEQ ID NO: 13, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 13.

40-43. (canceled)

44. The composition od of,
(a) wherein the AAV expression cassette further comprises a Kozak sequence;
(b) wherein the AAV expression cassette further comprises a Woodchuck hepatitis virus posttranscriptional regulatory element (WPRE);
(c) wherein the AAV expression cassette further comprises a polyadenylation signal;
and/or (d) wherein the expression cassette is present in an AAV vector selected from the group consisting of AAV2, AAV 8, AAV2/5, and AAV 2/8.

45. The composition of claim 44,
(a) wherein the Kozak sequence comprises the nucleotide sequence of GCCGCCACC, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of GCCGCCACC;
(b) wherein the WPRE comprises the nucleotide sequence of SEQ ID NO: 15, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 15;
(c) wherein the polyadenylation signal comprises the nucleotide sequence of SEQ ID NO: 16, or a nucleotide sequence having about 85%, 86%, 87%, 88%, 89%, 90%, 91%, 92%, 93%, 94%, 95%, 96%, 97%, 98%, or about 99% nucleotide sequence identity to the nucleotide sequence of SEQ ID NO: 16.

46-52. (canceled)

53. An AAV vector particle comprising the composition of claim 35.

54. An isolated cell comprising the AAV particle of claim 53.

55. A pharmaceutical composition comprising the AAV composition of claim 35.

56. (canceled)

57. The pharmaceutical composition of claim 55, which is formulated for intraocular administration.

58. The pharmaceutical composition of claim 57, wherein the intraocular administration is selected from the group consisting of intravitreal or subretinal, subvitreal, subconjuctival, sub-tenon, periocular, retrobulbar, suprachoroidal, and/or intrascleral administration.

59-62. (canceled)


SEQUENCE LISTING (40 sequences)

SEQ ID NO: 1 - DNA, 972 bp, Homo sapiens
  source 1..972: mol_type = unassigned DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 2 - DNA, 918 bp, Homo sapiens
  source 1..918: mol_type = unassigned DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 3 - DNA, 939 bp, Homo sapiens
  source 1..939: mol_type = unassigned DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataa

SEQ ID NO: 4 - DNA, 912 bp, Mus musculus
  source 1..912: mol_type = unassigned DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtga

SEQ ID NO: 5 - DNA, 1029 bp, Mus musculus
  source 1..1029: mol_type = unassigned DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaaagctgtagaggaaccccttaacgcatttaaagagtcaaaaggaatgatgaatgacgaatag

SEQ ID NO: 6 - DNA, 996 bp, Mus musculus
  source 1..996: mol_type = unassigned DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaaagctgtagaggaaccccttaacgaatag

SEQ ID NO: 7 - DNA, 975 bp, Mus musculus
  source 1..975: mol_type = unassigned DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtga

SEQ ID NO: 8 - DNA, 966 bp, Mus musculus
  source 1..966: mol_type = unassigned DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaaagctgtagaggaaccccttaacgcatttaaagagtcaaaaggaatgatgaatgacgaatag

SEQ ID NO: 9 - DNA, 2056 bp, Homo sapiens
  source 1..2056: mol_type = unassigned DNA
cctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccata

SEQ ID NO: 10 - DNA, 578 bp, Homo sapiens
  source 1..578: mol_type = unassigned DNA
tccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 11 - DNA, 584 bp, Human betaherpesvirus 5
  source 1..584: mol_type = unassigned DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 12 - DNA, 1667 bp, synthetic construct
  misc_feature 1..1667: note = Description of Artificial Sequence: Synthetic polynucleotide
  source 1..1667: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacag

SEQ ID NO: 13 - DNA, 810 bp, Homo sapiens
  source 1..810: mol_type = unassigned DNA
agatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatggatcctgagtaccttctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcc

SEQ ID NO: 14 - skipped

SEQ ID NO: 15 - DNA, 542 bp, Woodchuck hepatitis virus
  source 1..542: mol_type = unassigned DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 16 - DNA, 215 bp, synthetic construct
  misc_feature 1..215: note = Description of Artificial Sequence: Synthetic polynucleotide
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 17 - DNA, 130 bp, synthetic construct
  misc_feature 1..130: note = Description of Artificial Sequence: Synthetic polynucleotide
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 18 - DNA, 130 bp, synthetic construct
  misc_feature 1..130: note = Description of Artificial Sequence: Synthetic polynucleotide
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19-20 - skipped (2 entries)

SEQ ID NO: 21 - DNA, 5292 bp, Homo sapiens
  source 1..5292: mol_type = unassigned DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 22 - DNA, 5234 bp, Homo sapiens
  source 1..5234: mol_type = unassigned DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 23 - DNA, 5259 bp, Homo sapiens
  source 1..5259: mol_type = unassigned DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 24 - DNA, 2589 bp, Mus musculus
  source 1..2589: mol_type = unassigned DNA
agcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaggaaaaaaaaaaaaagctatagttgtaacatatgtattccagacctctggtttaaaggcaaaagaaaaaaaatctacagtgtttcttctcatgttttctgatcggaggcatgacaaagcaagactgaaatctgaactgtgtctcctgcatggcaacacgtgtctccgtcaggccctcgcaaggcccggggagggggttctacgcctcttgtctctttgttgcatgctgaacactcatcgccttcctactgtatcctgcctcctgcagcctccctcttcctcctcctcttcctcttcctcctcttcctcctcctcctcctcttcctccaagtttgaaaggtcaaacaaaactaccacattccctacccagttagaagaaaaccaccgtcctgacagttgtgatcgcatggagtacttttagattattagcacctgtttttacctcgtttgtgggcgtgtttgtatgtgcacatgtatgaagtcggcacatgcaccttctgtatgggcagaggcgtggcatctacagaagagcagatgccaactttgtgcttttagtgaatacattaaaaaaaaaaaaccaacggtccttattgagtggaattctatttgatgcaaatatttgagctctttaagactttaaaactagataatgtgccaagcttttaggactgctcaccagtgccctctgaagaaacaccagtactttttcctgtttgtgtaataaaggcatatttgtatttg

SEQ ID NO: 25 - DNA, 2710 bp, Mus musculus
  source 1..2710: mol_type = unassigned DNA
agcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaaagctgtagaggaaccccttaacgcatttaaagagtcaaaaggaatgatgaatgacgaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaggaaaaaaaaaaaaagctatagttgtaacatatgtattccagacctctggtttaaaggcaaaagaaaaaaaatctacagtgtttcttctcatgttttctgatcggaggcatgacaaagcaagactgaaatctgaactgtgtctcctgcatggcaacacgtgtctccgtcaggccctcgcaaggcccggggagggggttctacgcctcttgtctctttgttgcatgctgaacactcatcgccttcctactgtatcctgcctcctgcagcctccctcttcctcctcctcttcctcttcctcctcttcctcctcctcctcctcttcctccaagtttgaaaggtcaaacaaaactaccacattccctacccagttagaagaaaaccaccgtcctgacagttgtgatcgcatggagtacttttagattattagcacctgtttttacctcgtttgtgggcgtgtttgtatgtgcacatgtatgaagtcggcacatgcaccttctgtatgggcagaggcgtggcatctacagaagagcagatgccaactttgtgcttttagtgaatacattaaaaaaaaaaaaccaacggtccttattgagtggaattctatttgatgcaaatatttgagctctttaagactttaaaactagataatgtgccaagcttttaggactgctcaccagtgccctctgaagaaacaccagtactttttcctgtttgtgtaataaaggcatatttgtatttg

SEQ ID NO: 26 - DNA, 2677 bp, Mus musculus
  source 1..2677: mol_type = unassigned DNA
agcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaaagctgtagaggaaccccttaacgaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaggaaaaaaaaaaaaagctatagttgtaacatatgtattccagacctctggtttaaaggcaaaagaaaaaaaatctacagtgtttcttctcatgttttctgatcggaggcatgacaaagcaagactgaaatctgaactgtgtctcctgcatggcaacacgtgtctccgtcaggccctcgcaaggcccggggagggggttctacgcctcttgtctctttgttgcatgctgaacactcatcgccttcctactgtatcctgcctcctgcagcctccctcttcctcctcctcttcctcttcctcctcttcctcctcctcctcctcttcctccaagtttgaaaggtcaaacaaaactaccacattccctacccagttagaagaaaaccaccgtcctgacagttgtgatcgcatggagtacttttagattattagcacctgtttttacctcgtttgtgggcgtgtttgtatgtgcacatgtatgaagtcggcacatgcaccttctgtatgggcagaggcgtggcatctacagaagagcagatgccaactttgtgcttttagtgaatacattaaaaaaaaaaaaccaacggtccttattgagtggaattctatttgatgcaaatatttgagctctttaagactttaaaactagataatgtgccaagcttttaggactgctcaccagtgccctctgaagaaacaccagtactttttcctgtttgtgtaataaaggcatatttgtatttg

SEQ ID NO: 27 - DNA, 1928 bp, Mus musculus
  source 1..1928: mol_type = unassigned DNA
cccgggcagcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaaaaaaaaaa

SEQ ID NO: 28 - DNA, 2647 bp, Mus musculus
  source 1..2647: mol_type = unassigned DNA
agcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaaagctgtagaggaaccccttaacgcatttaaagagtcaaaaggaatgatgaatgacgaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaggaaaaaaaaaaaaagctatagttgtaacatatgtattccagacctctggtttaaaggcaaaagaaaaaaaatctacagtgtttcttctcatgttttctgatcggaggcatgacaaagcaagactgaaatctgaactgtgtctcctgcatggcaacacgtgtctccgtcaggccctcgcaaggcccggggagggggttctacgcctcttgtctctttgttgcatgctgaacactcatcgccttcctactgtatcctgcctcctgcagcctccctcttcctcctcctcttcctcttcctcctcttcctcctcctcctcctcttcctccaagtttgaaaggtcaaacaaaactaccacattccctacccagttagaagaaaaccaccgtcctgacagttgtgatcgcatggagtacttttagattattagcacctgtttttacctcgtttgtgggcgtgtttgtatgtgcacatgtatgaagtcggcacatgcaccttctgtatgggcagaggcgtggcatctacagaagagcagatgccaactttgtgcttttagtgaatacattaaaaaaaaaaaaccaacggtccttattgagtggaattctatttgatgcaaatatttgagctctttaagactttaaaactagataatgtgccaagcttttaggactgctcaccagtgccctctgaagaaacaccagtactttttcctgtttgtgtaataaaggcatatttgtatttg

SEQ ID NO: 29 - DNA, 4201 bp, Homo sapiens
  source 1..4201: mol_type = unassigned DNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 30 - DNA, 4580 bp, Homo sapiens
  source 1..4580: mol_type = unassigned DNA
actcacggccgctctccctcctcgctccgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgagtccttccggtgttcagtattcttgtctttgtgagtgcgtcccggggccgcctcggggcctgcctgccctcctgccaaagcctggaagaggattgaatggaccccagggtttggaaacaacctacagcatttgagcccctcacgtaggttttagagacgtacaatttttgtttgccctggctcagaaggagccggtgtaaggttgagataaaattccatatagacaactgagtttggatctcggctctgctgctttgtagctgtgggagttcaaacagcacctctttgagacttgggccccgcgtcggcacaatggggcaggaatagtccctgccagggtgactgtgtggattcaaggaggttggaagcactgagcccacgcttggcactgggtaggccatcactgaggcttgtgtctttccttcctgccctgaccctgcatagctgagtctcgtggcctctcaccactgatgggatctcggacacccggctcaccctcctcctggggcctcactttccttccccaccaaatgcagacatctaatgcttgtcctgtaatatccttgccagatgactgatgtcaagtgctggtttatagtgggtgctcaataaatgttagtttcctttctatccctccattagatcattaatgatattagtgctaattatgtaataaagttaaggcataaatatgttaa

SEQ ID NO: 31 - DNA, 4603 bp, Homo sapiens
  source 1..4603: mol_type = unassigned DNA
gcagccctcgctccctcccccggggcgcgcaggcgggcgctcgctcgcagagaagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgagtccttccggtgttcagtattcttgtctttgtgagtgcgtcccggggccgcctcggggcctgcctgccctcctgccaaagcctggaagaggattgaatggaccccagggtttggaaacaacctacagcatttgagcccctcacgtaggttttagagacgtacaatttttgtttgccctggctcagaaggagccggtgtaaggttgagataaaattccatatagacaactgagtttggatctcggctctgctgctttgtagctgtgggagttcaaacagcacctctttgagacttgggccccgcgtcggcacaatggggcaggaatagtccctgccagggtgactgtgtggattcaaggaggttggaagcactgagcccacgcttggcactgggtaggccatcactgaggcttgtgtctttccttcctgccctgaccctgcatagctgagtctcgtggcctctcaccactgatgggatctcggacacccggctcaccctcctcctggggcctcactttccttccccaccaaatgcagacatctaatgcttgtcctgtaatatccttgccagatgactgatgtcaagtgctggtttatagtgggtgctcaataaatgttagtttcctttctatccctccattagatcattaatgatattagtgctaattatgtaataaagttaaggcataaatatgttaa

SEQ ID NO: 32 - DNA, 4213 bp, Homo sapiens
  source 1..4213: mol_type = unassigned DNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggtacagtccttggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 33 - DNA, 4031 bp, Mus musculus
  source 1..4031: mol_type = unassigned DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 34 - DNA, 3377 bp, Mus musculus
  source 1..3377: mol_type = unassigned DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 35 - DNA, 4043 bp, Mus musculus
  source 1..4043: mol_type = unassigned DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 36 - DNA, 3845 bp, Mus musculus
  source 1..3845: mol_type = unassigned DNA
aagctcccctgccgcgggcagcctcttgcccactggagtctaaggactggccgggtgagaggccgagaccagggggcgatcggccgccacttccccagtccaccttaagaggaccaagtagccagcccgccgcgccgacctcagaaaaacaagtttgcgcaaagtggtgcgcggccagcctctgggcagagggagcggtgcttccaccgcctggcagccctgcgcgcggcggcgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 37 - DNA, 3389 bp, Mus musculus
  source 1..3389: mol_type = unassigned DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 38 - DNA, 3020 bp, Mus musculus
  source 1..3020: mol_type = unassigned DNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacagataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 39 - DNA, 3820 bp, Mus musculus
  source 1..3820: mol_type = unassigned DNA
aagctcccctgccgcgggcagcctcttgcccactggagtctaaggactggccgggtgagaggccgagaccagggggcgatcggccgccacttccccagtccaccttaagaggaccaagtagccagcccgccgcgccgacctcagaaaaacaagtttgcgcaaagtggtgcgcggccagcctctgggcagagggagcggtgcttccaccgcctggcagccctgcgcgcggcggcgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataa

SEQ ID NO: 40 - DNA, 3574 bp, Mus musculus
  source 1..3574: mol_type = unassigned DNA
cagcccctactggactcagcctcccttgcccagggcatgggatttacagcacacagaagaagtgggactgaaggacagacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataa